(19) United States
(12) Patent Application Publication
(10) Pub. No.: US 2026/0224801 A1
(43) Pub. Date: Aug. 6, 2026

(54) MIXING SYRINGE ASSEMBLIES AND METHODS OF MIXING CONSTITUENT MATERIALS

(71) Applicant: CLEARSTREAM TECHNOLOGIES LIMITED, Enniscorthy, County Wexford (IE)

(72) Inventors: Ruben Ramirez, Mesa, AZ (US); Charles D. Shermer, Raleigh, NC (US); Victor Bell, Dublin (IE); Jordan Addison, Gilbert, AZ (US); Matthew Cullen, Wicklow (IE)

(73) Assignee: CLEARSTREAM TECHNOLOGIES LIMITED, Enniscorthy, County Wexford (IE)

(21) Appl. No.: 19/154,973

(22) PCT Filed: Mar. 8, 2024
(86) PCT No.: PCT/US2024/019157

§ 371 Date: Aug. 8, 2025

(51) Int. Cl. A61M 5/14; A61M 5/19; A61M 5/31; A61N 5/10

(52) U.S. Cl. CPC A61M 5/1409; A61M 5/19; A61N 5/1007; A61M 2005/3128; A61N 2005/1021

(57) ABSTRACT

In one embodiment, a mixing syringe assembly for mixing two constituent materials includes a barrel body, a plunger, a sealing device, and one or more mixing masses. The plunger is slidably positioned within the barrel body. The sealing device divides the barrel body into a first constituent material section and a second constituent material section. The one or more mixing masses are positioned within one of the first constituent material section and the second constituent material section. The sealing device is operable to adjust from a sealing configuration to a non-sealing configuration to allow for a first constituent material from the first constituent material section to move into the second constituent material section upon movement of the plunger along the barrel body and be mixed therein with the one or more mixing masses.

### CROSS-REFERENCE TO RELATED APPLICATIONS

The present application claims the benefit of U.S. Provisional Application No. 63/489,497, filed Mar. 10, 2023, U.S. Provisional Application No. 63/498,614, filed Apr. 27, 2023, U.S. Provisional Application No. 63/498,686, filed, Apr. 27, 2023, and U.S. Provisional Application No. 63/509,355, filed Jun. 21, 2023, the entireties of which are hereby incorporated by reference.

### BACKGROUND

### Field

The present disclosure generally relates to mixing syringe assemblies and methods of mixing constituent materials and, more particularly, mixing syringe assemblies and methods of mixing constituent materials for radiation spacers.

### Technical Background

Prostate cancer is the most common non-skin cancer diagnosed in men. Radiation therapy is an excellent treatment option for prostate cancer. However, radiation exposure can cause unintended side effects in adjacent non-targeted tissue. A radiation spacer, such as a radio protective spacer can be implanted to avoid collateral radiation and minimize injury to nearby tissues by providing a space between the target tissue and non-targeted tissues at risk.

Conventional radiation spacers may include gelling materials, which are delivered as a liquid and allowed to cure. However, constituent components of the gel may begin to gel on contact with one another, which may make delivery difficult. Moreover, constituent components may need to remain separate until time of use, otherwise they may degrade over time due to limited stability.

### SUMMARY

Embodiments of the present disclosure are directed to various mixing syringe assemblies, which may maintain separation of constituent materials until time for use of the mixed constituent materials. Embodiments are also directed to accessories which may be used in conjunction with one or more of the mixing syringe assemblies as described herein.

In one embodiment, a mixing syringe assembly for mixing two constituent materials includes a barrel body, a plunger, a sealing device, and one or more mixing masses. The plunger is slidably positioned within the barrel body. The sealing device divides the barrel body into a first constituent material section and a second constituent material section. The one or more mixing masses are positioned within one of the first constituent material section and the second constituent material section. The sealing device is operable to adjust from a sealing configuration to a non-sealing configuration to allow for a first constituent material from the first constituent material section to move into the second constituent material section holding a second constituent material upon movement of the plunger along the barrel body and be mixed therein with the one or more mixing masses.

In another embodiment, a mixing syringe assembly for mixing multiple constituent components includes a first mixing syringe subassembly and a second mixing syringe subassembly coupled to the first mixing syringe subassembly. Each mixing syringe subassembly includes a barrel body, a plunger, a sealing device, and one or more mixing masses. The plunger is slidably positioned within the barrel body. The sealing device divides the barrel body into a first constituent material section and a second constituent material section. The one or more mixing masses are positioned within one of the first constituent material section and the second constituent material section. The sealing device is operable to adjust from a sealing configuration to a non-sealing configuration to allow for a first constituent material from the first constituent material section to move into the second constituent material section upon movement of the plunger along the barrel body and be mixed therein with the one or more mixing masses.

In yet another embodiment, a method of mixing constituent materials positioned within a mixing syringe assembly includes advancing a plunger within a barrel body toward a sealing device separating the barrel body into a first constituent material section and a second constituent material section; adjusting the sealing device from a sealing configuration to a non-sealing configuration thereby providing a flow path between the first constituent material section and the second constituent material section; pushing with the plunger a first constituent material from the first constituent material section into the second constituent material section, the second constituent material section holding a second constituent material and one or more mixing masses; and shaking the mixing syringe assembly to combine the first constituent material with the second constituent material.

In yet another embodiment, a mixing syringe assembly for mixing two constituent materials includes, a barrel body, a plunger, and a sealing device. The a barrel body includes a first barrel body portion and a second barrel body portion which the first barrel body portion is slidably disposed in the second barrel body portion, the first barrel body portion defining a first constituent material section and the second barrel body portion defining a second constituent material section. The plunger is slidably positioned within the first barrel body portion. The sealing device includes a piercing member coupled to a distal end of the first barrel body portion and a canister positioned within the second barrel body portion. The canister includes a first pierceable film and a second pierceable film and a second constituent material is position within the canister between the first pierceable film and the second pierceable film. The piercing member is configured to pierce the first pierceable film and the second pierceable film to release the second constituent material. A first constituent material is positioned within the first constituent material section and advancing the plunger releases the first constituent material in to the second constituent material section.

In yet another embodiment, a mixing syringe assembly includes a first mixing syringe subassembly, a second mixing syring subassembly, and a fixture. The first mixing syringe subassembly and t second mixing syringe subassembly are coupled to one another and incude a first barrel body portion having a flange and a second barrel body portion, wherein the first barrel body portion is slidably disposed within the second barrel body portion. The fixture is coupled to the flange of the first mixing syringe subassembly and the second mixing syringe subassembly such that movement of the first barrel body portion of the first mixing syringe subassembly and the first barrel body portion of the second mixing syringe subassembly are tied to one another via the fixture.

In yet another embodiment, a mixing syringe assembly includes a first mixing syringe subassembly, a second mixing syringe, a fixture, a two-piece piston insert. Each mixing syringe subassembly includes a barrel body having a flange and a plunger slidably positioned within each of the first barrel body portions. The fixture is coupled to the flange of the first mixing syringe subassembly and the second mixing syringe subassembly. The two-piece piston insert is coupled to each of the plungers, wherein the two-piece piston insert engages with the fixture to prevent withdrawal of the plungers from the barrel bodies.

Additional features and advantages of the aspects described herein will be set forth in the detailed description, which follows, and in part will be readily apparent to those skilled in the art from that description or recognized by practicing the aspects described herein, including the detailed description, which follows, the claims, as well as the appended drawings.

It is to be understood that both the foregoing general description and the following detailed description describe various aspects and are intended to provide an overview or framework for understanding the nature and character of the claimed subject matter. The accompanying drawings are included to provide a further understanding of the various aspects, and are incorporated into and constitute a part of this specification. The drawings illustrate the various aspects described herein, and together with the description serve to explain the principles and operations of the claimed subject matter.

### BRIEF DESCRIPTION OF THE DRAWINGS

The embodiments set forth in the drawings are illustrative and exemplary in nature and not intended to limit the subject matter defined by the claims. The following detailed description of the illustrative embodiments can be understood when read in conjunction with the following drawings, wherein like structure is indicated with like reference numerals and in which:

FIG. 1 schematically depicts an embodiment of a syringe assembly for mixing constituent materials, according to one or more embodiments shown and described herein;

FIG. 2 schematically depicts a dual syringe assembly for mixing constituent materials, according to one or more embodiments shown and described herein;

FIG. 3A schematically depicts a sealing device including a valve in a closed configuration, according to one or more embodiments shown and described herein;

FIG. 3B schematically depicts the sealing device of FIG. 3A moved to an open configuration, according to one or more embodiments shown and described herein;

FIG. 3C schematically depicts after actuation of the sealing device of FIG. 3A, movement of constituent material from a first constituent material section into a second constituent material section and agitation thereof, according to one or more embodiments shown and described herein;

FIG. 3D schematically depicts dispensing of the combined first and second constituent materials from the mixing syringe assembly, according to one or more embodiments shown and described herein;

FIG. 4 schematically depicts a syringe assembly for mixing constituent materials, according to one or more embodiments shown and described herein;

FIG. 5A schematically depicts a sealing device including a gate valve in a closed configuration, according to one or more embodiments shown and described herein;

FIG. 5B schematically depicts the sealing device of FIG. 5A moved to an open configuration, according to one or more embodiments shown and described herein;

FIG. 5C schematically depicts a side view of the sealing device of FIG. 5A, according to one or more embodiments shown and described herein;

FIG. 5D schematically depicts another side view of the sealing device of FIG. 5A, according to one or more embodiments shown and described herein;

FIG. 6 schematically depicts a dual syringe assembly for mixing constituent materials, according to one or more embodiments shown and described herein;

FIG. 7 schematically depicts assembly of a sealing device to the syringe assembly of FIG. 6, according to one or more embodiments shown and described herein;

FIG. 8 schematically depicts an incorporated mounting structure for a sealing device for the dual syringe assembly of FIG. 6, according to one or more embodiments shown and described herein;

FIG. 9A schematically depicts a first constituent material positioned within a first constituent material section and a second constituent material positioned within a second constituent material section of the mixing syringe assembly of FIG. 4, according to one or more embodiments shown and described herein;

FIG. 9B schematically depicts opening of a gate valve of the mixing syringe assembly of FIG. 9A to allow first constituent material to flow into the second constituent material section, according to one or more embodiments shown and described herein;

FIG. 9C schematically depicts dispensing the combined constituent materials of FIG. 9B, according to one or more embodiments shown and described herein;

FIG. 10A schematically depicts an embodiment of a syringe assembly for mixing constituent materials, according to one or more embodiments shown and described herein;

FIG. 10B schematically depicts a sealing device of the syringe assembly of FIG. 10A moving over a protrusion to move the sealing device to an open configuration, according to one or more embodiments shown and described herein;

FIG. 10C schematically depicts an axial view of the sealing device of FIG. 10B according to one or more embodiments shown and described herein;

FIG. 11A schematically depicts a syringe assembly for mixing constituent materials having a plunger in a first position, according to one or more embodiments shown and described herein;

FIG. 11B schematically depicts the plunger of FIG. 11A moved to a second position, according to one or more embodiments shown and described herein;

FIG. 11C schematically depicts the plunger and a first barrel body portion move to a third position, according to one or more embodiments shown and described herein;

FIG. 12 schematically depicts a dual syringe assembly for mixing constituent materials, according to one or more embodiments shown and described herein;

FIG. 13A schematically depicts a perspective view of the dual syringe assembly of FIG. 12, according to one or more embodiments shown and described herein;

FIG. 13B schematically depicts a cross-section view of the dual syringe assembly of FIG. 12 having one or more constituent materials positioned therein, according to one or more embodiments shown and described herein;

FIG. 13C schematically depicts depression of plungers of the dual syringe assembly of FIG. 13B from a first position to a second position to move diluent from first barrel body portions to second barrel body portions, according to one or more embodiments shown and described herein;

FIG. 13D schematically depicts the plungers and first barrel body portions of FIG. 13C moving from the second position to a third position to move combined constituent materials from the second barrel body portions out of the dual syringe assembly, according to one or more embodiments shown and described herein;

FIG. 14A schematically depicts a perspective view of a second barrel portion for use in a dual syringe assembly, according to one or more embodiments shown and described herein;

FIG. 14B schematically depicts a cross-section of the second barrel portion of FIG. 14A, according to one or more embodiments shown and described herein;

FIG. 15A schematically depicts a sealing device within a dual barrel syringe assembly, according to one or more embodiments shown and described herein;

FIG. 15B schematically depicts a portion of the sealing device in isolation, according to one or more embodiments shown and described herein;

FIG. 15C schematically depicts a cross-section of the portion of the sealing device of FIG. 15B, according to one or more embodiments shown and described herein;

FIG. 16 schematically depicts a dual syringe assembly for mixing constituent materials, according to one or more embodiments shown and described herein;

FIG. 17A schematically illustrates depression of a first barrel body to a third or fourth position to puncture a capsule positioned within a second barrel body, according to one or more embodiments shown and described herein;

FIG. 17B schematically illustrates a hook device engaging a capsule of FIG. 17A, according to one or more embodiments shown and described herein;

FIG. 17C schematically illustrates a more detailed view of the hook device of FIG. 17B engaging the capsule, according to one or more embodiments shown and described herein;

FIG. 18 depicts a flow chart illustrating a method of mixing at least two constituent materials within a mixing syringe assembly, according to one or more embodiments shown and described herein;

FIG. 19A schematically depicts a two-piece alignment fixture for use in a dual mixing syringe assembly, according to one or more embodiments shown and described herein;

FIG. 19B schematically depicts a placement of the two-piece alignment fixture of FIG. 19A around a proximal end of a dual mixing syringe assembly, according to one or more aspects shown and described herein;

FIG. 19C depicts the two-piece alignment fixture assembled around the proximal end of the dual mixing syringe assembly of FIG. 19B, according to one or more embodiments shown and described herein;

FIG. 20A is schematically depicts a two-piece alignment fixture for use in a dual mixing syringe assembly, according to one or more embodiments shown and described herein;

FIG. 20B schematically depicts a portion of the two-piece alignment fixture of FIG. 20A, according to one or more aspects shown and described herein;

FIG. 20C schematically depicts a portion of the two-piece alignment fixture coupled to a dual mixing syringe assembly, according to one or more embodiments shown and described herein;

FIG. 21 schematically depicts the two-piece alignment fixture assembled to the dual mixing syringe assembly of FIG. 20C and a two-piece piston insert, according to one or more embodiments shown and described herein;

FIG. 22A schematically depicts one half of the two-piece piston insert of FIG. 21, according to one or more embodiments shown and described herein;

FIG. 22B schematically depicts a frontal view of the two-piece piston insert of FIG. 22A, according to one or more embodiments shown and described herein;

FIG. 22C schematically depicts a perspective view of the two-piece piston insert of FIG. 22A, according to one or more embodiments shown and described herein;

FIG. 22D schematically depicts a frontal view of a modified two-piece piston insert, according to one or more embodiments shown and described herein;

FIG. 23 schematically depicts a plunger for use with a two-piece piston insert, according to one or more embodiments shown and described herein; and

FIG. 24 schematically depicts a two-piece piston insert partially mounted to the plunger of FIG. 23, according to one or more embodiments shown and described herein.

### DETAILED DESCRIPTION

The present disclosure is generally directed to mixing syringe assemblies for mixing at least two constituent materials. More particularly the present application is directed to mixing syringe assemblies configured to delivering a radiation spacer. For example, radiation spacers may be formed of one or more hydrogel materials, which may be delivered to a desired location, such as within a balloon or on its own and cured in place to block or substantial block radiation, which may be unintentionally directed toward healthy tissue instead of targeted/diseased tissue. During mixing of the hydrogel for forming a radiation spacer, multiple materials may need to be combined just prior to and/or at delivery of the radiation spacer into a target location within the body. To maintain quality of material, powder components may need to be kept dry or un-constituted prior to application. However, during use, powder components may need to be hydrated or mixed with a diluent. It may be difficult to determine proper mixing volumes, maintain sterility, and prevent unwanted early mixing using traditional methods. Moreover, traditional mixing may increase unwanted air bubbles present within the resulting hydrogel, which may obscure imaging. Embodiments of the present disclosure may provide constituent materials in prepackaged, pre-measured, ready-to-use syringe assemblies, which, as will be described in greater detail herein, provide improved mixing with reduced air bubbles, maintains sterility, and/or improves delivery. Additional accessories which may provide for improved control and/or functionality are also disclosed herein.

Particular embodiments of a mixing syringe assembly include a barrel body, a plunger, a sealing device, and, in some embodiments, one or more mixing masses. The sealing device is operable to divide a barrel body into a first constituent material section and a second constituent material section, thereby maintaining separation of a first constituent material in the first constituent material section and a second constituent material within the second constituent material section until the seal is moved from a closed configuration to an open configuration. Various sealing devices will be described throughout the present disclosure. In embodiments including the one or more mixing masses, the one or more mixing masses may be positioned in either the first constituent material section or the second constituent material section and may be used to mix the first and second constituent materials once combined in the second constituent material section. As will be described in greater detail below, use of the one or more mixing masses assists with thoroughly mixing the first and second constituent materials while reducing introduction of air bubbles as may generally occur without use of one or more mixing masses. These and additional embodiments and benefits will be described in greater detail below.

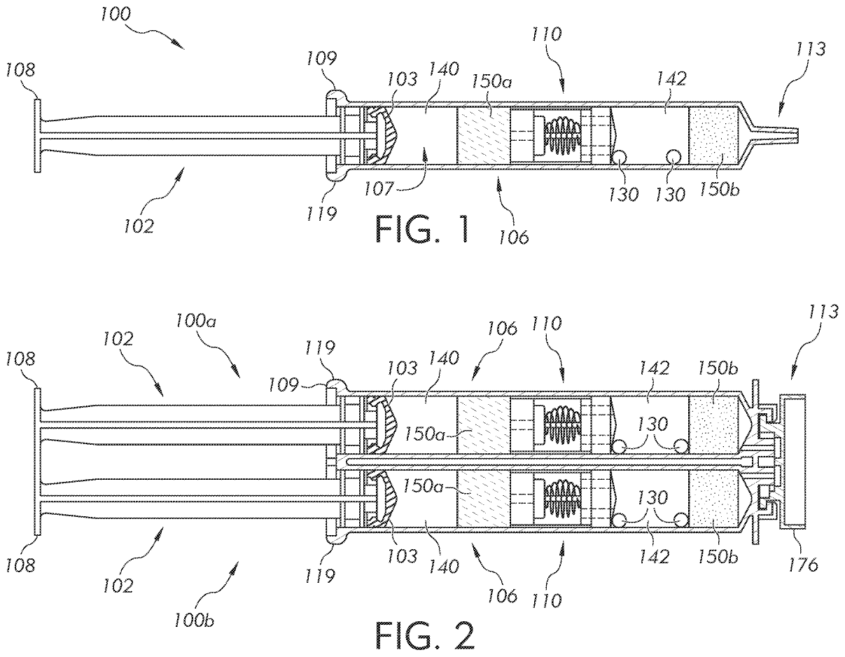

Turning now to the drawings, FIG. 1 depicts an illustrative mixing syringe assembly 100. The mixing syringe assembly 100 may be used for combining two or more constituent materials as will be described in greater detail herein. In particular, the mixing syringe assembly 100 may be used to mix or combine two or more constituent components of a hydrogel for use as a radiation spacer or component thereof. The mixing syringe assembly 100 generally includes a plunger 102, a barrel body 106, and a sealing device 110. In some embodiments, the mixing syringe assembly 100 further includes one or more mixing masses 130. It is noted that a mixing syringe assembly 100 may include a greater or fewer number of components without departing from the scope of the present disclosure.

The plunger 102 may be slidably disposed in a lumen 107 of the barrel body 106 and may form a fluidic seal with the barrel body 106 via a seal 103 at a distal end of the plunger 102. For example, the seal 103 may be a rubber or similar, conformable material for forming a fluid-tight seal with the barrel body 106. At a proximal end of the plunger 102 may be a pusher flange 108 for a user to engage for advancing the plunger 102 along the barrel body 106.

The barrel body 106 may be generally hollow and have plunger-receiving end 109 at a proximal end for receiving the plunger 102 and a fluid delivery opening 113 at a distal end, which may be coupled to a material delivery cannula (not shown, which may include a manifold, delivery needle or the like). In embodiments, the fluid delivery opening 113 may be axially positioned with a centerline of the barrel body 106 and/or plunger 102. However, in other embodiments, the fluid delivery opening 113 may be offset from a centerline of the barrel body 106 and/or plunger 102. Such offset may be useful in preventing the one or more mixing masses 130 from blocking the fluid delivery opening 113.

The sealing device 110 separates the barrel body 106 into a first constituent material section 140 and a second constituent material section 142. The first constituent material section 140 and the second constituent material section 142 may initially take up the same volume or substantially same volume as depicted. However, in some embodiments, initial volumes may differ from one another. As will be described in greater detail, a first constituent material 150a may be positioned initially within the first constituent material section 140 and a second constituent material 150b, different from the first constituent material 150a, may be positioned within the second constituent material section 142. As depicted in the illustrated embodiment, the first constituent material section 140 may be positioned proximal to the second constituent material section 142.

The first constituent material 150a may be a liquid for hydrating the second constituent material 150b, which may be a powder or particulate. For example, the first constituent material 150a may be saline, water, deionized water, or the like. As noted above, the second constituent material 150b may be a powder or particulate material such as but not limited to albumin, polyethylenimine (PEI), an amine containing polyethylene glycol (PEG) or protein, an N-hydroxysuccinimide (NHS) ester component such as PEG-(SS)2, PEG-(SS)4, PEG-(SS)8, PEG-(SG)4, PEG-(SG)8, and/or the like. In some aspects, molecular weights of the PEG components may range from about 2,000 to about 100,000. The powder or particular material may be biodegradable and/or bioabsorbable. As used herein, “biodegradable” and/or “bioabsorbable” refers to a compound that can be absorbed by the surrounding or local tissue of a subject and/or degraded and absorbed by the tissue of the subject.

The powder or particulate material can be composed of various crosslinking substances of varying amounts designed to allow the hydrogel to last a specific amount of time in situ before degrading. In aspects, the hydrogel components may be selected based on a degradation time that corresponds to the length of anticipated radiation therapy. In aspects the length of anticipated radiation therapy, and thus the targeted time for hydrogel degradation is up to 18 months, for example from the range of about 0 months to about 18 months, including about 1 month, 2 months, 3 months, 4 months, 5 months, 6 months, 7 months, 8 months, 9, months, 10 months, 11 months, 12 months, 13 months, 14 months, 15 months, 16 months, 17 months, and 18 months. It should be understood that the time is merely a rough guide generally used to target appropriate formulation of the hydrogel.

The sealing device 110 may take on several different forms. In general though, the various sealing devices 110 described herein may move or are otherwise modified to selectively provide a flow path between the first constituent material section 140 and the second constituent material section 142 to allow for the first constituent material from the first constituent material section 140 to move into the second constituent material section 142 to allow for mixing of the first constituent material 150a with the second constituent material 150b.

The one or more mixing masses 130 may initially be positioned within either the first constituent material section 140 of the second constituent material section 142. It is noted that in embodiments, placing the one or more mixing masses 130 within the second constituent material section 142 may be beneficial in that a flow path large enough to allow the one or more mixing masses 130 through need not be created to move the one or more mixing masses 130 from the first constituent material section 140 to the second constituent material section 142. The one or more mixing masses 130 may be any suitable material to assist in mixing materials such as stainless steel, delron, plastic polycarbonate, composite, aluminum, low carbon steel, chemical-resistant PTFE, composite, titanium, moisture resistant polyethylene, magnetic material, etc. In one or more mixing masses 130, may be any shape such as spherical, cylindrical, cubic, ellipsoid, or any other regular or irregular shape. The one or more mixing masses 130 may include any number of masses such as two or more, three or more, four or more etc. The one or more mixing masses 130 may be identical to one another or different from one another. The one or more mixing masses 130 may have various sizes such as up to 7 mm in diameter, such as up to 6 mm in diameter, such as up to 5 mm in diameter, such as up to 4 mm in diameter, such as up to 3 mm in diameter, such as 7 mm or less in diameter. In various embodiments, the one or more mixing masses 130 may have designs cut or etched into its surface, which may enhance mixing.

Referring now to FIG. 2, in various embodiments, a mixing syringe assembly 100 may include one or more mixing syringe subassemblies, such as to provide a dual barrel syringe. For example, FIG. 2 illustrates a dual-mixing syringe assembly including a first mixing syringe subassembly 100a and a second mixing syringe subassembly 100b. Each subassembly 100a, 100b is substantially identical to the mixing syringe assembly 100 described with respect to FIG. 1. In particular, each subassembly 100a may include a plunger 102, a barrel body 106, and a sealing device 110. In some embodiments, the subassemblies 100a, 100b further include one or more mixing masses 130. Accordingly, the above description is applicable to the present embodiments and will not be repeated for brevity. However, in the present embodiment, the plungers 102 may be coupled to one another via a common pusher flange 108 as depicted. However, it is contemplated and possible that each plunger 102 may be independently operable from one another such that the plungers 102 are not coupled via a common pusher flange 108. In some embodiments, as will be described in greater detail herein, attached to the plungers 102 may be a two-piece piston insert, which may assist in advancing the plungers 102 simultaneously and/or providing improved stability.

In some embodiments, the barrel bodies 106 may be rigidly coupled to one another. For example, the adjacent barrel bodies may be integrally formed or coupled to another via a bracket, welding, adhesive or the like. In some embodiments, a manifold 176 may couple the fluid delivery openings to respective delivery needles or cannulas (not shown) for delivery of fluid. It is noted that in various embodiments, it may be desirable to keep mixed constituent materials from the first subassembly 100a and the second subassembly 100b separate until the point of delivery at a target delivery location, such as within a subject. Accordingly, dual needle assemblies and/or coaxial needle assemblies such as described in International Patent Application No. PCT/US2021/023171, entitled “Multi-Component Sealant Delivery Systems Incorporating Quarter Turn Connectors,” filed Mar. 19, 2021, the entirety of which is hereby incorporated by reference. For example, while the first mixing syringe subassembly 100a may include the first constituent material 150a and the second constituent material 150b, the second mixing syringe subassembly 100b may hold a different first constituent material 150a and/or a different second constituent material 150b from that held in the first mixing syringe subassembly 100a. For example, a second constituent material 150b of the first mixing syringe subassembly 100a may hold Peg 8, having 20 kmw, while the second constituent material 150b of the second mixing syringe subassembly may hold Peg 8 having 15 kmw. The first constituent materials 150a may also be the same or different and may include, but are not limited to water saline, deionized water, or like. In embodiments, the resulting mixtures or solutions within each of the first and second subassemblies 100a, 100b, may gel or solidify on contact with one another. Accordingly, it may be desirable to only allow contact at the moment of delivery, such as at the end of a dual-lumen cannula, such as disclosed in International Patent Application No. PCT/US2021/023171, entitled “Multi-Component Sealant Delivery Systems Incorporating Quarter Turn Connectors,” filed Mar. 19, 2021, the entirety of which is hereby incorporated by reference.

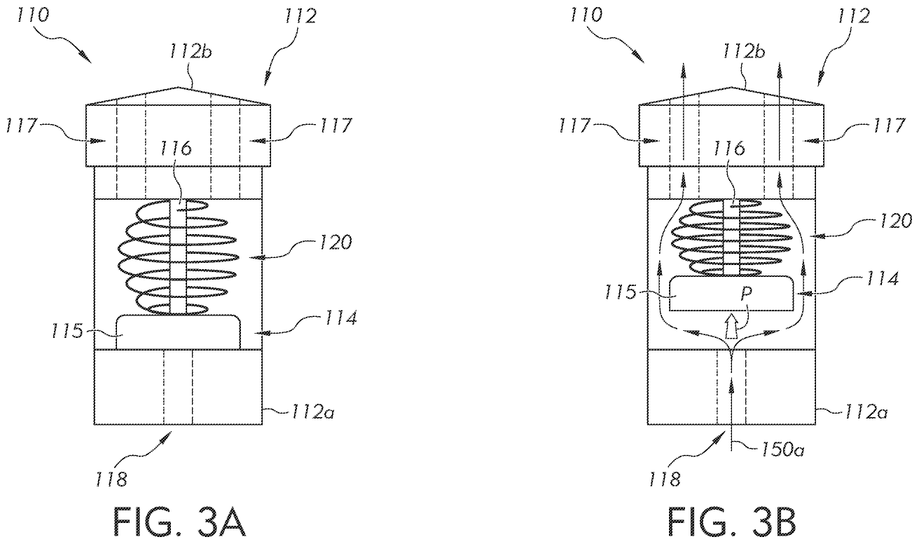

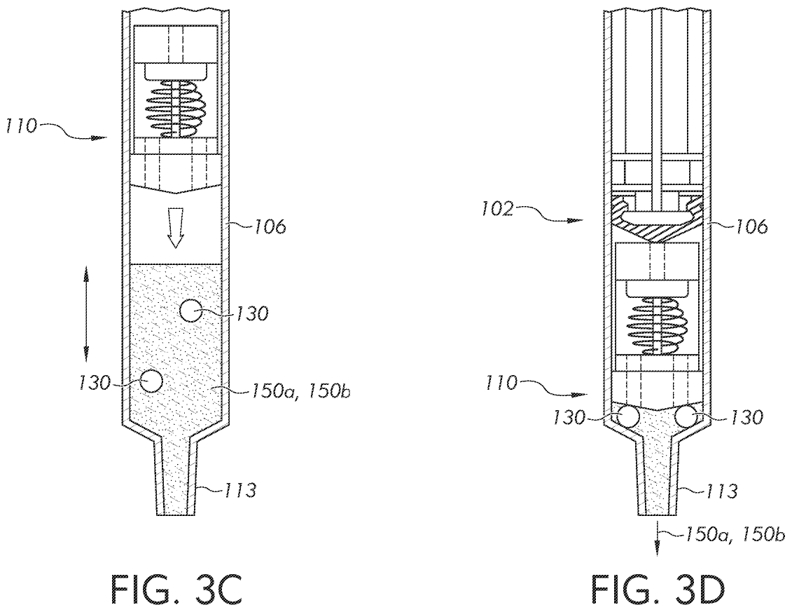

FIGS. 3A and 3B generally depict a sealing device 110 according to at least one embodiment, which may be used and is depicted in the above described assemblies. For example, the sealing device 110 may be a one-way valve. The one-way valve may include a housing 112, a valve closure 114, and a spring 120. It is noted that the one-way valve may have additional or different components without departing from the scope of the present disclosure.

The housing 112 may be size and shaped to fit within the barrel body 106 described above. In particular, the housing 112 may form a seal 103 with the barrel body 106 so as to be fluid tight when the valve closure 114 is moved to a closed configuration. In embodiments, the housing 112 may be formed of and/or coated with a sealing material such as rubber or other conformable materials suitable for forming a seal.

The housing 112 may include a proximal portion 112a and a distal portion 112b. The proximal portion 112a may be positioned within the barrel body 106 as closer to the plunger 102 than the distal portion 112b. The proximal portion 112a may define one or more flow paths therethrough such as proximal flow path 118. The distal portion 112b may also define one or more flow paths therethrough, such as distal flows paths 117. It is noted that, while one flow path is illustrated in the proximal portion 112a and two flow paths are illustrated in the distal portion 112b, either portion may have any number of fluid flow paths.

The valve closure 114 may be a device configured to be selectively moved to seal off the proximal flow path 118. For example, the valve closure 114 may have a valve disk 115. The valve disk 115 in the closed configuration may be seated against the proximal portion 112a to block or seal 103 off the proximal flow path 118. In some embodiments, the valve disk 115 may have a guiding rod 116, which may be configured to move along a path (not shown) such as formed within the distal portion 112b to maintain alignment of the valve disk 115 relative to the proximal flow path 118. The valve disk 115 may be formed of any sealing material such as rubber or like.

The spring 120, such as a helical spring, may bias the valve closure 114, such as the valve disk 115, to the closed configuration such that is it seated against the proximal portion 112a to block or seal off the proximal flow path 118. In the open configuration, the helical spring 120 may be compressed, such as by advancing the plunger 102 distally, allowing the valve closure 114 to move distally thereby unsealing the proximal flow path 118 and allowing first constituent material 150a from the first constituent material section 140 to move through the sealing device 110, such as through the proximal flow path 118 and the distal flow path(s) 117 into the second constituent material section 142.

For example, a first constituent material 150a may be positioned within the first constituent material section 140 and a second constituent material may be positioned within the second constituent section 142. The first constituent material may be a fluid (e.g., saline) and the second constituent material may be a powder or particulate, as described above. The plunger 102 may be advanced along the barrel body 106. Movement of the plunger 102 toward the sealing device 110 may actuate the sealing device 110 from a closed configuration to an open configuration. For example, the first constituent material 150a would exert pressure P on the sealing device 110 upon movement of the plunger toward the sealing device 110, particularly the valve disk 115 in the present embodiment. The spring 120, in response to the pressure compresses, allowing the valve closure 114 to move from the closed configuration (shown in FIG. 3A) to the open configuration (shown in FIG. 3B). As the plunger 102 is further advanced along the barrel body 106, the first constituent material 150a flows into the second constituent material section 142 through the sealing device 110. Once the first constituent material 150a and the second constituent material 150b are both within the second constituent material section 142 (depicted in FIG. 3C), the reduction in fluid pressure allows the sealing device 110 to move back to the closed configuration via the spring 120 bias. The mixing syringe assembly 100 may then be agitated (e.g., shaken) to incorporate that first and second constituent materials 150a, 150b, such as illustrated in FIG. 3C. As noted above, the one or more mixing masses 130 may assist in agitation while reducing introduction of air bubbles.

After mixing the plunger 102 may be advanced further and push the sealing device 110 along the barrel body 106, such that the sealing device 110 slides distally and together the sealing device 110 and plunger 102 expel the combined constituent materials 150a, 150b out of the fluid delivery opening 113, such as generally depicted in FIG. 3D. After delivery, the one or more mixing masses 130 may remain in the second constituent material section 142 so as not to pass out of the fluid delivery opening 113. For example, the one or more mixing masses 130 may be sized so as to be unable to exit the barrel body 106 through the fluid delivery opening 113. It is noted that in various embodiments, the liquid constituent material may be placed in the second constituent material section 142 and the powder or particulate component may be placed initially in the first constituent material section.

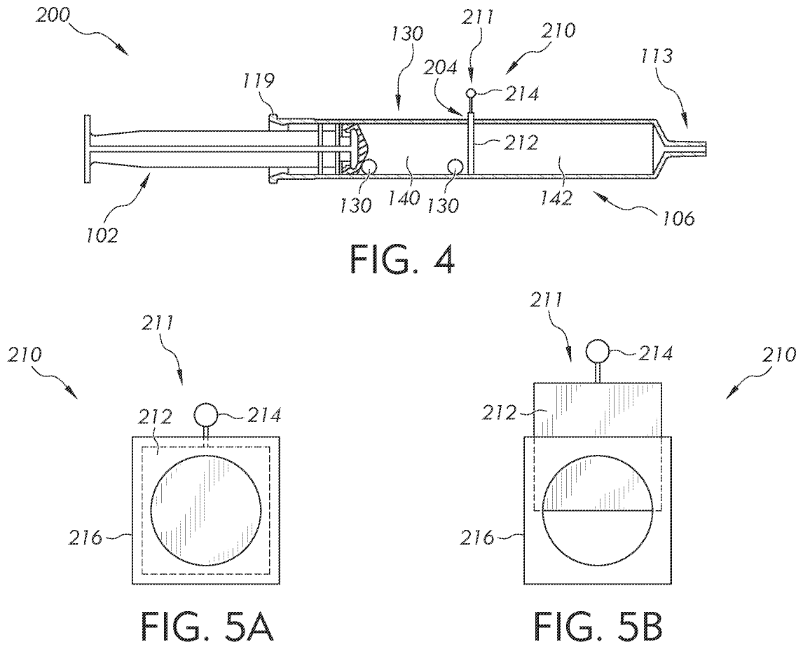

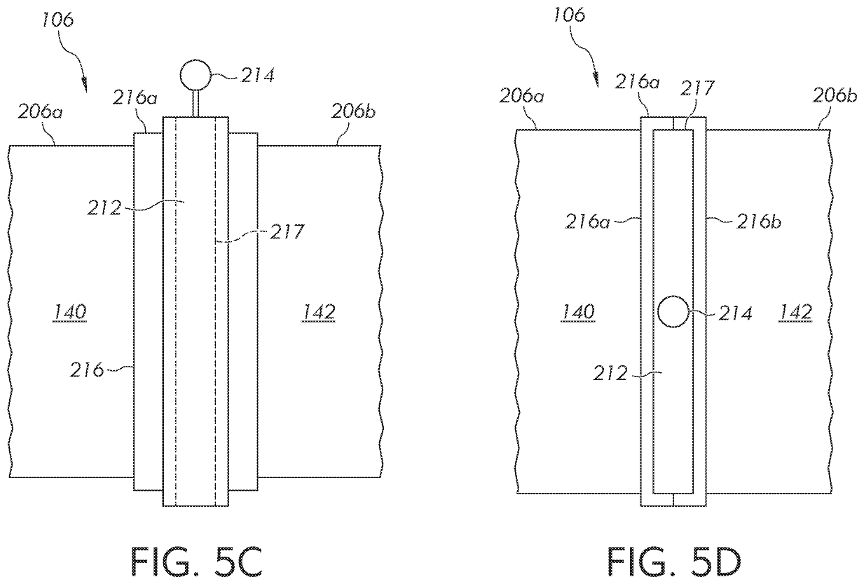

FIG. 4 illustrates an alternative embodiment of a mixing syringe assembly 200. Similar to the mixing syringe assembly 100 above, the mixing syringe assembly 200 generally includes a plunger 102, a barrel body 106, and a sealing device 210. In some embodiments, the mixing syringe assembly 200 further includes one or more mixing masses 130. It is noted that a mixing syringe assembly 200 may have more or less components without departing from the scope of the present disclosure. The plunger 102, barrel body 106 and mixing masses 130 are as described above unless otherwise noted or apparent. Accordingly, for brevity the description fo the plunger 102, barrel body 106, and mixing masses 130 will not be repeated. However, in the present embodiment, the sealing device 110 includes a gate valve 211 configured to slide in a radial direction relative to a centerline of the barrel body 106 to move the sealing device 110 from a closed configuration (depicted in FIG. 5A) to an open configuration (depicted in FIG. 5B).

The gate valve 211 may generally include a slidable gate 212 which may be inserted through an opening 204 formed in the barrel body 106. In embodiments the slidable gate 212 may be a substrate, which may be formed or at least partially formed of one or more sealing materials (e.g., rubber, delron, plastic, polycarbonate, composite, or the like) to seal 103 both the opening 204 and the first constituent material section 140 from the second constituent material section 142. In embodiments, the gate valve 211 may include a handle 214 to allow a user to grasp the gate 212 and move the gate valve 211 between the open and closed configurations.

Referring to FIG. 5A-5D, in embodiments, the gate valve 211 may include a housing 216 coupled to the barrel body 106. The housing 216 may have a square or rectangular shape as shown or may have a rounded shape. Other shapes are contemplated and possible. The housing 216 may be made from any suitable material such as, but not limited to Delron, plastic, polycarbonate, composite or the like. For example, the gate valve 211 may include a housing 216 that divides and couples portions of the barrel body 106. For example, the barrel body 106 may include a first barrel body portion 206a and a second barrel body portion 206b and the housing 216 may include a first housing portion 216a coupled to a distal end of the first barrel body portion 206a and a second housing portion 216b coupled to a proximal end of the second barrel body portion 206a. Accordingly, the first constituent material section 140 may be within the first barrel body portion 206a and the second constituent material section 142 may be in the second barrel body portion 206b. The first and second housing portions 216a, 216b may be coupled to one another so as to form a slot 217 between the first and second housing portions 216a, 216b for receiving the slideable gate 212. Barrel body portions 206a, 206b and/or the first and second housing portions 216a, 216b may be coupled to one another via any conventional means such as welding, adhesive, or the like. In embodiments, it is noted that the first and second housing portions 216a, 216b may be integrally formed with one another and coupled to the first and second barrel body portions 206a, 206b. Alternatively, it is contemplated that the barrel body portions 206a, 206b and the housing portions 216a, 216b may all be integrally formed within one another or portions thereof.

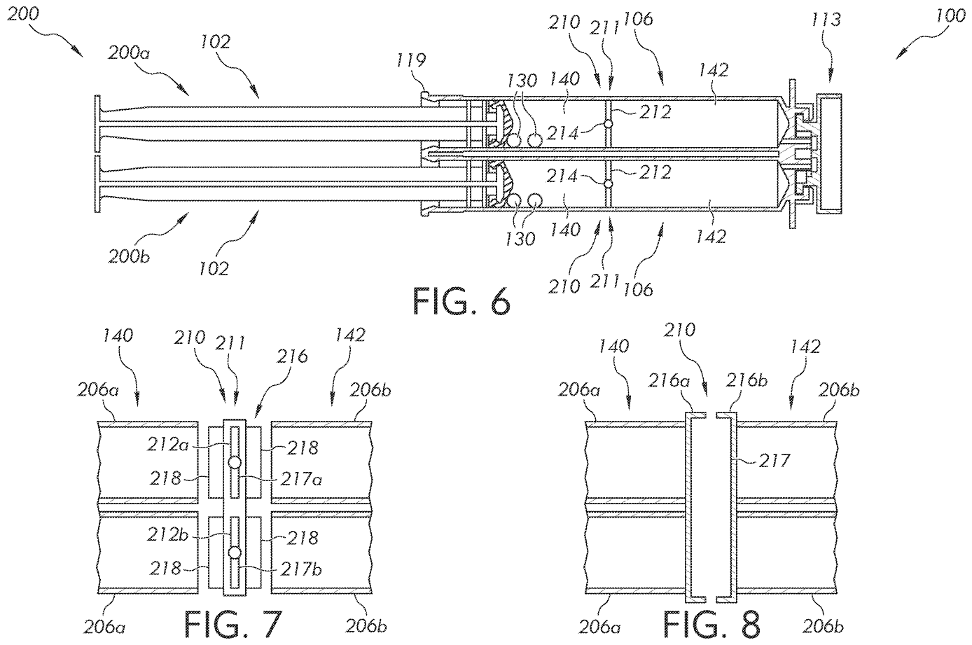

FIG. 6 illustrates a mixing syringe assembly with a dual barrel syringe similar to the embodiment shown in FIG. 2 above, but with both mixing syringe subassemblies 200a, 200b including a gate valve 211. Referring now to FIG. 7, in some embodiments, there may be a single housing 216, which couples to each barrel body portion 206a, 206b of the first and second mixing syringe subassemblies 200a, 200b. For example, in FIG. 7 there may be a single housing 216 including a first slidable gate 212a and a second slideable gate 212b, such that the single valve housing 216 defines two slots 217a, 217b corresponding to each subassembly 200a, 200b. In embodiments, the housing 216 may further include coupling extensions 218 configured to be received within each barrel body portion 206a, 206b and seal thereto. The coupling extensions may define flow paths therethrough to allow for plunger 102 traversal.

Referring to FIG. 8, in some embodiments, a first housing portion 216a may be coupled to the distal end of both first barrel body portions 206a and a second housing portion 216b may be coupled to both second barrel body portions 206b, such that only a single slot 217 is provided. In such embodiments, slidable gate (not shown) may block and open fluid communication between the first constituent material portion and the second constituent material portion. Seals or O-rings may be positioned within the housing to provide a leak proof seal between barrels.

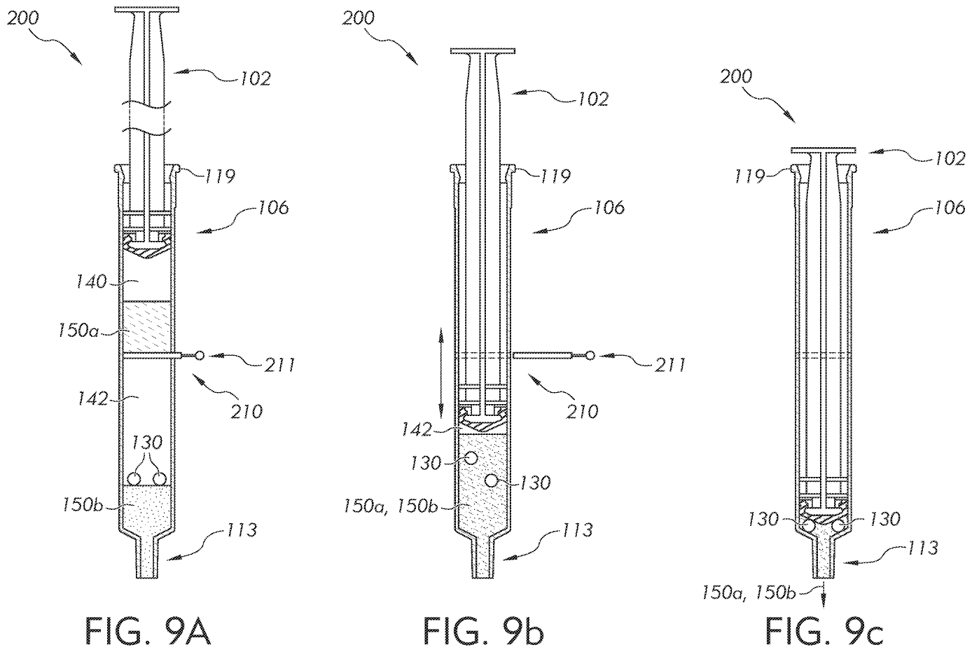

Referring now to FIGS. 9A-9B, during use, such as once mixing of the first constituent material 150a positioned within the first constituent material section 140 and the second constituent material 150b within the second constituent material section 142 is desired, the gate valve 211 may be opened thereby allowing the first constituent material 150a to flow into the second constituent material section 142. The mixing syringe assembly 100 may then be agitated (e.g., shaken) to incorporate that first and second constituent materials 150a, 150b. As noted above, the one or more mixing masses 130 may assist in agitation while reducing introduction of air bubbles. After mixing, the plunger 102 may be advanced further, such as illustrated in FIG. 9C, to expel the combined constituent materials 150a, 150b out of the fluid delivery opening 113 or respective delivery fluid opening in a dual-barrel syringe assembly as described above. It is noted that in various embodiments, the liquid constituent material may be placed in the second constituent material section 142 and the powder or particulate component may be placed initially in the first constituent material section 140.

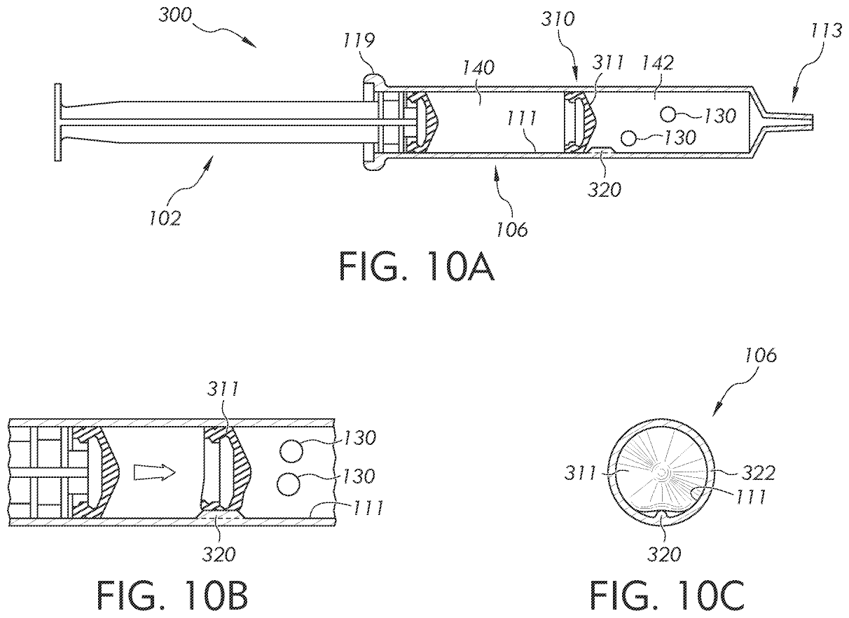

FIGS. 10A-10C illustrate an alternative embodiment of a mixing syringe assembly 300. Similar to the mixing syringe assemblies above, the mixing syringe assembly 300 generally includes a plunger 102, a barrel body 106, and a sealing device 310. In some embodiments, the mixing syringe assembly 300 further includes one or more mixing masses 130. It is noted that a mixing syringe assembly 300 may have more or less components without departing from the scope of the present disclosure. The plunger 102, barrel body 106 and mixing masses 130 are as described above unless otherwise noted or apparent. However, in the present embodiment, the sealing device 310 includes a radially deformable valve 311 and a protrusion 320 such as formed on or extending from an interior surface 111 of the barrel body 106.

As in the embodiments above, the deformable valve 311 may be initially positioned between and fluidically isolate the first constituent material section 140 and the second constituent material section 142 from one another. The deformable valve 311 may be a plug formed of the conformable material (such as rubber or the like) so as to adequately seal against unwanted leakage between the first and second constituent material sections 140, 142. The deformable valve 311 may be slidable along the barrel body 106 in response to pressure, thereby providing a longitudinally moveable seal. For example, during use, the plunger 102 may be advanced. Pressure, such as fluid pressure, may build up within the first constituent material section 140 and cause the deformable valve 311 to advance distally.

As noted above, a protrusion 320 may be formed on or otherwise extend from the interior surface 111 of the barrel body 106. The protrusion 320 may cause the deformable valve to deform and lift away from or otherwise remove its seal to the interior surface 111 as depicted in FIGS. 10B and 10C. Once lifted, flow paths may be formed such as on either side of the protrusion 320 allowing the first constituent material (not shown) to flow from the first constituent material section 140 to the second constituent material section 142. As in embodiments above, the mixing syringe assembly 300 may then be agitated (e.g., shaken) to incorporate that first and second constituent materials. As noted above, the one or more mixing masses 130 may assist in agitation while reducing introduction of air bubbles. After mixing, the plunger 102 may be advanced further and contact the deformable valve, then together the deformable valve and the plunger 102 may be advance distally to expel the combined constituent materials out of the fluid delivery opening 113. It is noted that in various embodiments, the liquid constituent material may be placed in the second constituent material section 142 and the powder or particulate component may be placed initially in the first constituent material section 140. As in embodiments above, the present embodiment may be incorporated in to any of the dual syringe assemblies described herein. For example, the sealing device 110 of the present embodiment may replace the sealing device 110 of any of the embodiments described herein.

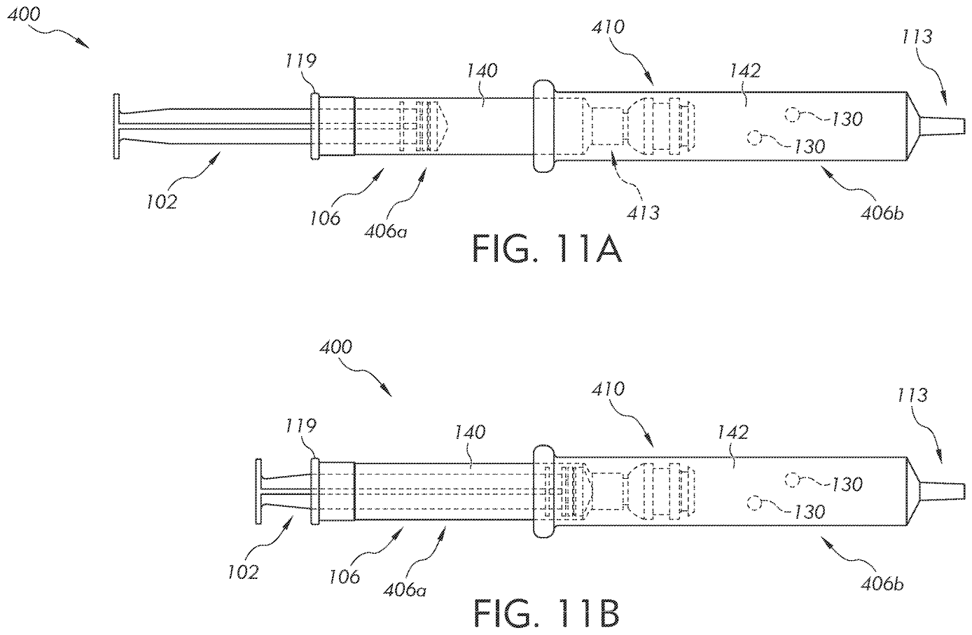

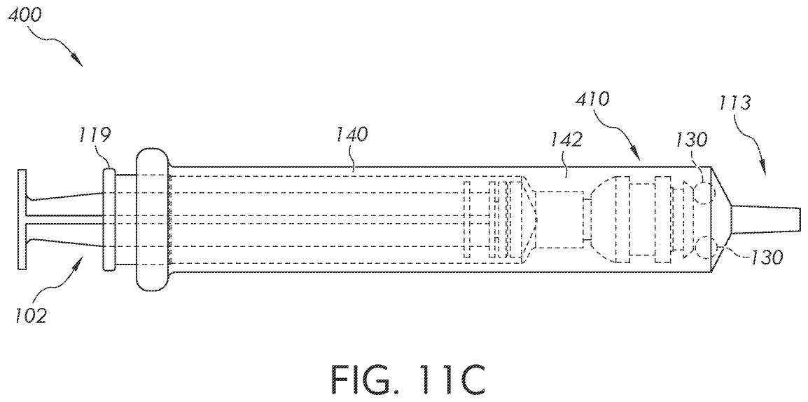

FIG. 11A-11C depict yet another alternative embodiment of a mixing syringe assembly 400. Similar to the mixing syringe assemblies above, the mixing syringe assembly 400 generally includes a plunger 102, a barrel body 106, and a sealing device 410. In some embodiments, the mixing syringe assembly 400 further includes one or more mixing masses 130. It is noted that a mixing syringe assembly 400 may have more or less components without departing from the scope of the present disclosure. The plunger 102, barrel body 106 and mixing masses 130 are as described above unless otherwise noted or apparent.

In the present embodiment, the barrel body 106 includes a first barrel body portion 406a and a second barrel body portion 406b, wherein the first barrel body portion 406a is slidably positioned within the second barrel body portion 406b such that the first barrel body portion 406a may telescope within the second barrel body portion 406b. That is, the first barrel body portion 406a may have a smaller diameter than the second barrel body portion 406b. For example, the first barrel body portion 406a may be a 5 ml barrel and the second barrel body portion 506b may be a 10 ml barrel, though other sizes are contemplated and possible. In the instant embodiment, the first barrel body portion 406a defines the first constituent material section 140 and the second barrel body portion 406b defines the second constituent material section 142.

The first barrel body portion 406a may include a fluid delivery opening 413 located at a distal end of the first barrel body portion 406a. In the present embodiment, the sealing device 410 may be coupled to the distal end of the first barrel body portion 406a such as around the fluid delivery opening 413. The sealing device 410 may both seal to the inner surface of the second barrel body portion 406b, such that material does not leak between the first barrel body portion 406a and the second barrel body portion 406b.

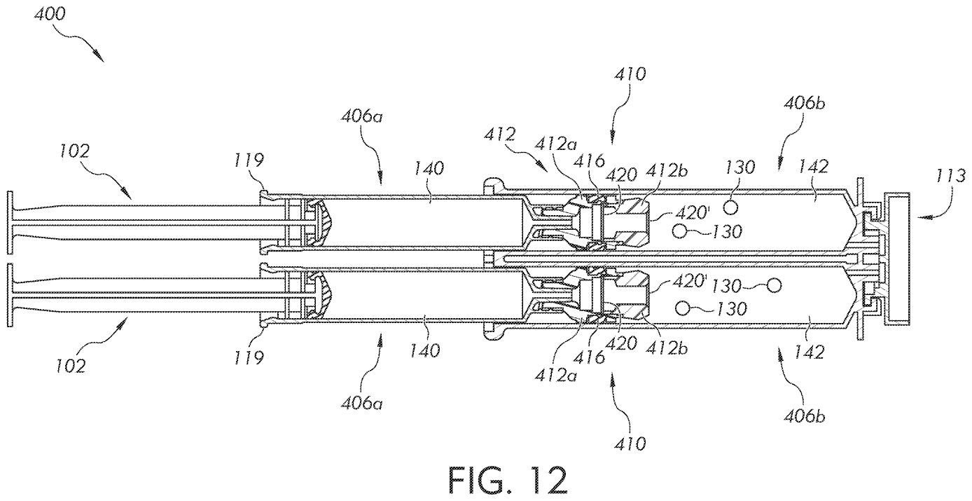

The sealing device 410 is best illustrated in FIG. 12, which also illustrates a dual barrel mixing syringe assembly similar to those described above. It is noted however, the sealing device 410 may be incorporated into either a single or dual barrel design. The sealing device 410 in the present embodiment generally includes a housing 412 which is couplable to the distal end of the first barrel body portion 406a as described above. For example, the housing 412 may be threadingly coupled to the distal end or the first barrel body portion 406a, though other fittings or attachments (e.g., press fits, interference fits, etc.) are contemplated and possible. In embodiments, the housing 412 may be coupled to the distal end of the first barrel body portion 406a in a fluid-tight manner including any number of sealing materials and/or seals (e.g., O-rings) to ensure a fluid-tight seal and prevent leakage. In embodiment, an outer seal 416 (such as an O-ring) is coupled to an outer surface of the housing 412 and engages an inner surface of the second barrel body portion 406b to provide a fluid-tight seal therewith. The housing 412 defines a flow path therethrough to allow first constituent material to flow from the first constituent material section 140 to the second constituent material section 142 when actuated.

In embodiments, the housing 412 may include proximal portion 412a and a distal portion 412b that couples to the proximal portion 412a, each defining a portion of the flow path. The proximal portion 412a and the distal portion 412b may be press fit together, threadingly engaged, or the like. Sandwiched between the proximal portion 412a and the distal portion 412b is a burstable film 420. The burstable film 420 may be a thin layer of film (e.g., plastic, foil, or the like) that extends across and blocks the flow path to prevent unwanted movement of first constituent material into the second constituent material section 142. In embodiments, there may be multiple burstable films positioned along the housing 412. For example, a second burstable film 420′ may be coupled to a distal end of the distal portion 412b. The films may be coupled to the housing 412 such as via adhesive, interlocks, heat sealing, heat staking, induction sealing, ultrasonic welding, etc.

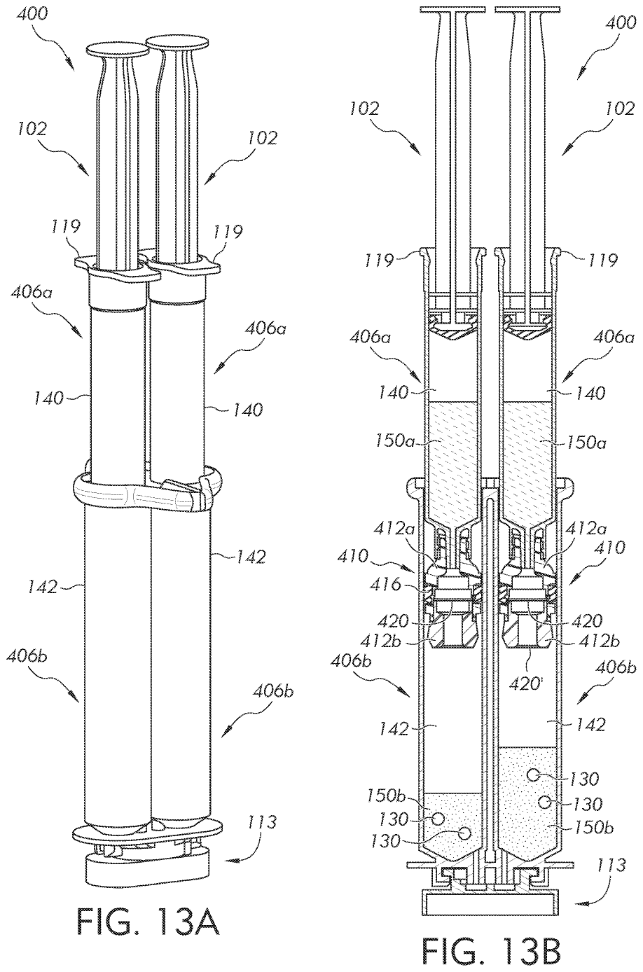

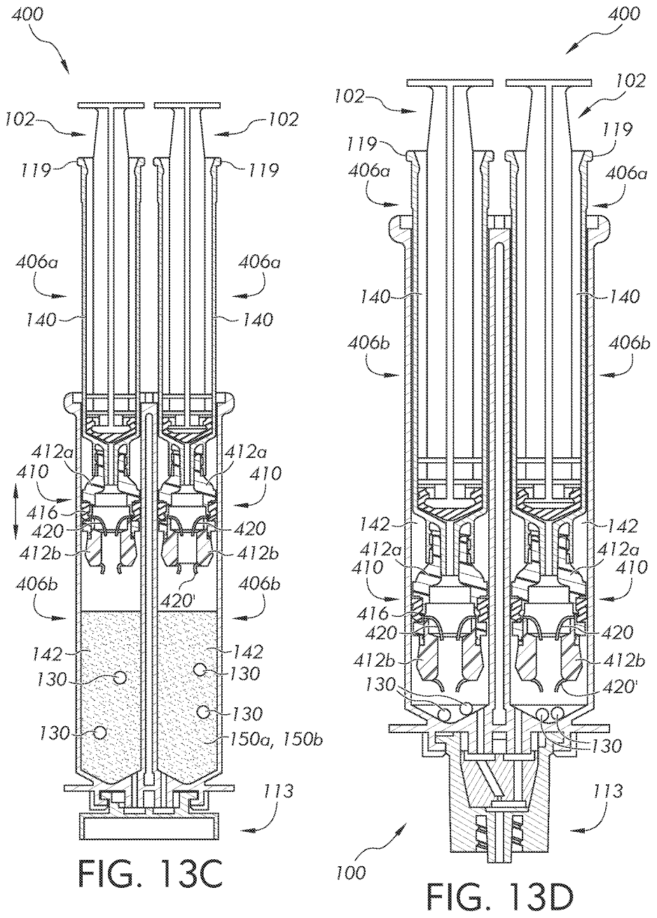

Referring to FIGS. 13A-13D, operation of the mixing syringe assembly 400 is generally depicted. FIG. 13A illustrates a perspective first of a dual syringe assembly version of the mixing syringe assembly 400. In the particularly illustrated embodiment, the second barrel body portions 406b may be coupled to one another and/or integrally formed with one another. Such allows for easier placement, control, and use of the mixing syringe assembly 400 when part of a dual barrel design as shown. FIG. 13B illustrates a cross-sectional view of FIG. 13A and depicts a first constituent material 150a positioned within respective first constituent material sections 140 and a second constituent material 150b positioned within respective second constituent material sections 142. Referring to FIG. 13C, in operation, the plunger(s) 102 of the assembly 400 may be advanced building fluid pressure within the first constituent material section 140 behind the burstable film 420 and/or 420′. Once the pressure exceeds a threshold of the burstable film, the burstable film 420, 420′ may burst, rip, or tear to allow first constituent material 150a to pass from the first constituent material section 140 through the sealing device 410 to the second constituent material section 142 within the second barrel portion 406a (e.g., of the respective sub-assembly). Once the first constituent material 150a enters the second constituent material section 142, the mixing syringe assembly 400 may then be agitated (e.g., shaken) to incorporate that first and second constituent materials as in embodiments above. As noted above, the one or more mixing masses 130 may assist in agitation while reducing introduction of air bubbles. After mixing, the plunger(s) 102 and the first barrel body portion 406a together with the sealing device 410 may be advanced together to expel the combined constituent materials out of the fluid delivery opening 113 (or respective opening in dual design). It is noted that in various embodiments, the liquid constituent material may be placed in the second constituent material section 142 and the powder or particulate component may be placed initially in the first constituent material section 140.

In embodiments including a dual barrel design as shown, one mixing syringe subassembly may include albumin and/or microparticles as the second constituent material 150b and the other mixing syringe subassembly may include PEG. Other combinations are contemplated and possible. After mixing with respective liquid constituent materials the now-liquid albumin and PEG solutions may be dispensed as described above to form a gel at the desired location.

In some embodiments, it is contemplated that the powder/particulate constituent material may be initially positioned between the first film 420 and the second film 420′ and released into the second constituent material section 142 via bursting the first and second films 420, 420′.

It is noted that in the embodiment above, while the system may be provided as completely assembled, in some embodiments, the different components may be provided individually and assembled prior to use.

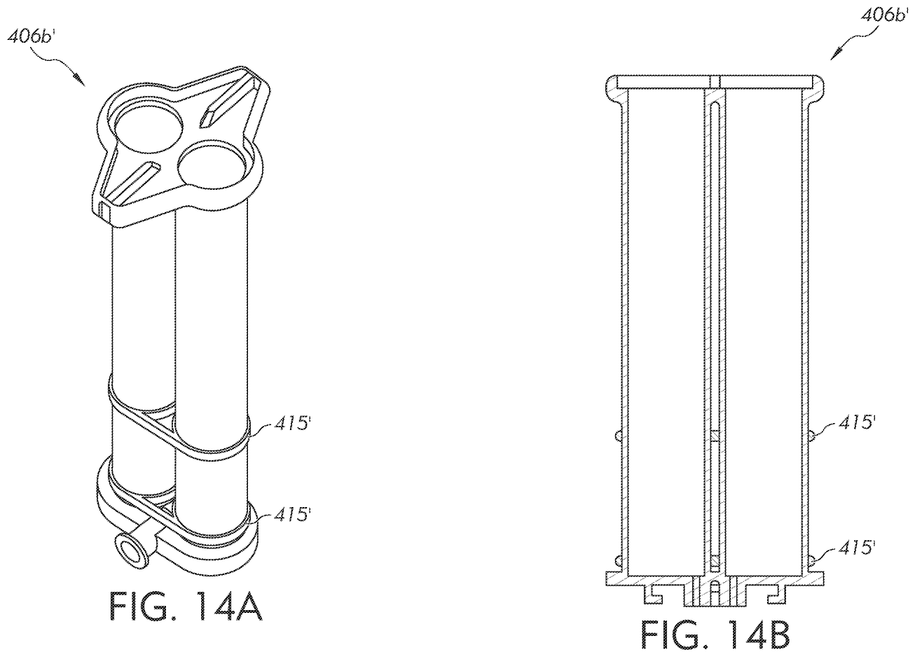

Referring now to FIGS. 14A and 14B, an alternative embodiment of the second barrel portion 406b′ is generally depicted. It is noted that the second barrel portion 406b′ is substantially similar to the second barrel body portion 406b described above, except as otherwise noted. In particular, a distal end of the second constituent material portion 142 of the second barrel portion 406b′ is flattened, as opposed to tapered as illustrated above. Such design allows the first barrel body portion 406a along with the sealing device 410, and plunger 102 to be more fully compressed to the distal end of the second barrel portion 406b′. Such may allow an increased amount of gel to be delivered, and may also increase the visual feedback to the user as the plunger 102/sealing device 410 will be fully at the distal end of the barrel with no or reduced gaps, such as where the second barrel portion 406′ is formed of a transparent or translucent material. Additionally, in the illustrated embodiment, marking bands 415′, such as two spaced marking bands 415′ are provided on the second barrel portion 406b′ and provide feedback to a user as to how much gel has been delivered. The most proximal marking band may indicate 50% of the gel has been delivered, for example, and the most distal marking may indicate all of the gel has been delivered. These marking bands 415′ will allow the user to establish how much gel is left to deliver by taking a quick look at the plunger(s) 102 position relative to the markings. The marking bands 415′ may be integrally formed with the second barrel portion 416b or may be separately coupled thereto. Accordingly, the marking bands, may be a rubber, plastic, or similar material

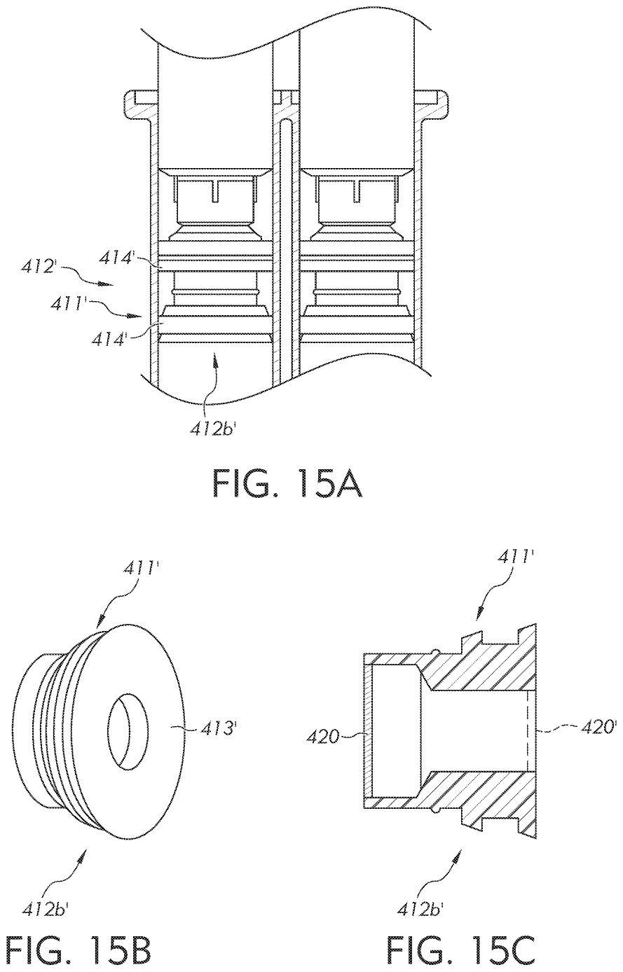

Referring now to FIG. 15A-15C, an alternative embodiment of the sealing device 410′ is depicted. In particular, the sealing device 410′ includes, as above, a proximal portion 412a′ and a distal portion 412b′ similar to those described above. However, in the present embodiment, the distal portion 412b′ is modified to reduce the extending drafted boss, depicted in the embodiment above, and widen the diameter of the housing 412′ at the distal portion 412b′ to provide a larger flat distal surface 413′. The housing 412′, such as the distal portion 412b′ has also been altered to allow an O-ring 414′ to be placed on it, reducing the potential for any backflow at the sides of the distal portion 412b′. That is an O-ring recess 411′ may be provided on the distal portion 412b′. In embodiments, an O-ring 414′ may be positioned on both the proximal portion 412a′ and the distal portion 412b′, thereby improving fluid tightness between the second barrel portion 406b′ and the sealing device 410′. The larger flat distal surface 413′ of the distal portion 412b′ will allow the sealing device 410′ to be pushed fully downward to the flat distal end of the second barrel portion 406b′, described above.

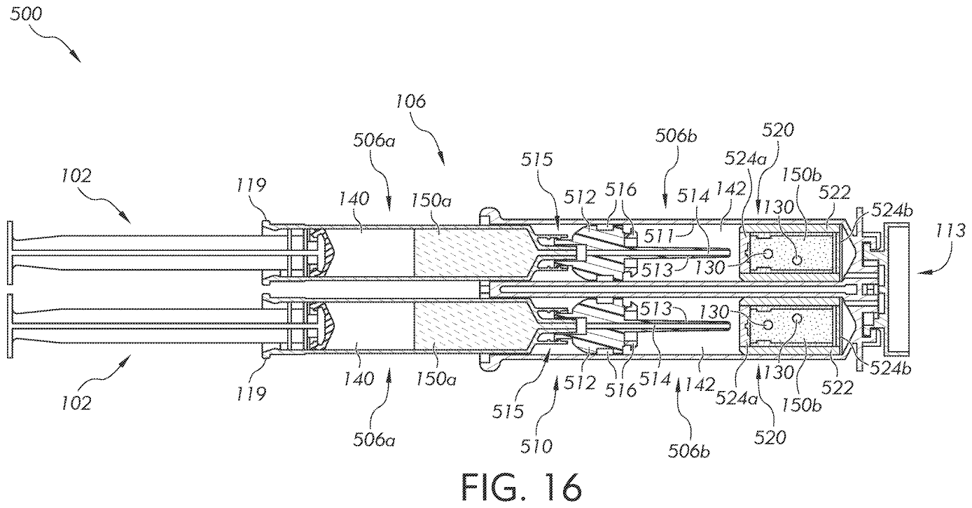

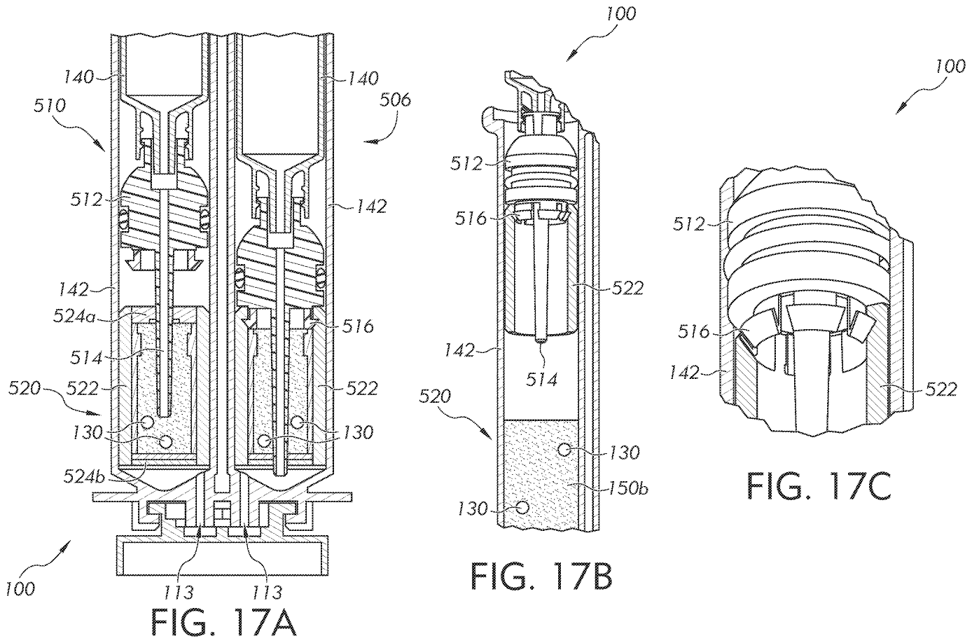

FIG. 16-17C depict yet another alternative embodiment of a mixing syringe assembly 500. Similar to the mixing syringe assemblies above, the mixing syringe assembly 500 generally includes a plunger 102, a barrel body 106, and a sealing device 510. In some embodiments, the mixing syringe assembly 500 further includes one or more mixing masses 130. It is noted that the mixing syringe assembly 500 may have more or less components without departing from the scope of the present disclosure. The plunger 102, barrel body 106, and mixing masses 130 are as described above unless otherwise noted or apparent.

As in the embodiments of FIGS. 11A-11C, the barrel body 506 includes a first barrel body portion 506a and a second barrel body portion 506b, wherein the first barrel body portion 506a is slidably positioned within the second barrel body portion 506b such that the first barrel body portion 506a may telescope with respect to the second barrel body portion 506. That is, the first barrel body portion 506a may have a smaller diameter than the second barrel body portion 506b. For example, the first barrel body portion 506a may be a 5 ml barrel and the second barrel body portion 506b may be a 10 ml barrel, though other sizes are contemplated and possible. In the instant embodiment, the first barrel body portion 506a defines the first constituent material section 140 and the second barrel body portion 506b defines the second constituent material section 142.

The first barrel body portion 506a may include a fluid delivery opening 515 located at a distal end of the of the first barrel body portion 506a. In the present embodiment, the sealing device 510 may be coupled to, such as partially coupled to, the distal end of the first barrel body portion 506a such as around the fluid delivery opening 515. The sealing device 510 may seal to the inner surface 511 of the second barrel body portion 506b such that material does not leak past the first barrel body portion 506a.

In the present embodiment, the sealing device 510 includes a piercing member 512 and a capsule 520. The piercing member 512 is couplable to the distal end of the first barrel body portion 506a. For example, the piercing member 512 may be threadingly coupled to the distal end of the first barrel body portion 506a, though other fittings or attachments (e.g., press fits, interference fits, etc.) are contemplated and possible. In embodiments, the piercing member 512 may be coupled to the distal end of the first barrel body portion 506a in a fluid-tight manner including any number of sealing materials and/or seals (e.g., O-rings) to ensure the fluid-tight and prevent leakage. The piercing member 512 may include an elongate, piercing nose 513 which may be used to pierce the capsule 520 described in greater detail below. In some embodiments, the piercing nose 513 may be generally straight and/or tapered to assist in piercing the capsule 520. In embodiment, an outer seal (such as an O-ring) may be coupled to an outer surface of the piercing member and engages an inner surface of the second barrel body portion 506b to provide a fluid-tight seal therewith. The piercing member 512 defines a flow path 514 therethrough to allow first constituent material 150a to flow from the first constituent material section 140 to the second constituent material section 142.

The capsule 520 is positioned within the second constituent material section 142, so as to be within the second barrel body portion 506b. The capsule 520 may include a canister body 522 a first pierceable film 524a forming a proximal surface of the canister body 522 and a second pierceable film 524b forming a distal surface of the canister body 522. In embodiments, the second constituent material 150b and/or the one or more mixing masses 130 may be positioned within the canister body 522. In some embodiments, the one or more mixing masses 130 may be positioned distal to canister body 522. The first and second pierceable films 524a, 524b may be any pierceable film such as plastic, foil, or the like.

During use, the first barrel body portion 506a may be advanced to pierce the first pierceable film 524a with the piercing member 512 as illustrated in FIG. 17A. The first barrel body portion 506a may be advanced further to pierce the second pierceable film 524b as also illustrated in FIG. 17A. In embodiments, the piercing member 512 may be include a hook device 516 configured to engage and couple to the canister body 522. For example, the hook device 516 may be positioned circumferentially around the piercing nose 513 of the piercing member 512. The hook device 516 may include one or more radially outward facing hooks, such as a plurality of radially outward facing hooks. When advanced to pierce the second pierceable film 524b, the one or more radially outward facing hooks may engage the capsule 520, such as the canister body 522 and become coupled thereto. For example, the canister body 522 may have a deformable sidewall that the one or more radially outward facing hooks may latch into. Once latched, the capsule 520 may be withdrawn, and the second constituent material 150b may fall from the canister into the second constituent material section 142. The plunger 102 may then be advanced into the first barrel body portion 506a and the first constituent material released 150a into the second constituent material section 142.

Once the first constituent material enters the second constituent material section 142, the mixing syringe assembly 100 may then be agitated (e.g., shook) to incorporate that first and second constituent materials. As noted above, the one or more mixing masses 130 may assist in agitation while reducing introduction of air bubbles. After mixing, the plunger 102 may be advanced further and the first barrel body portion 506a together with the sealing device 510 may be advanced together to expel the combined constituent materials out of the fluid delivery opening 113 (or respective opening in dual design). It is noted that while the present embodiment is illustrated as dual syringe embodiment, the sealing device 510 may be instead be incorporated in to a single syringe design.

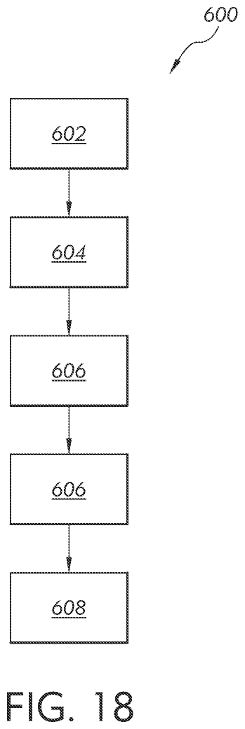

FIG. 18 generally depicts a flow chart illustrating a method 600 of mixing constituent materials, which may be applicable to any of the single and/or dual syringe embodiments as described herein. In particular, the method 600 includes at step 602 advancing a plunger 102 within a barrel body 106 toward a sealing device 110 separating the barrel body 106 into a first constituent material section 140 and a second constituent material section 142. Step 604 includes adjusting the sealing device 110 from a sealing configuration to a non-sealing configuration thereby providing a flow path between the first constituent material section 140 and the second constituent material section 142. Step 606 includes pushing with the plunger 102 a first constituent material 150a from the first constituent material section 140 into the second constituent material section 142, the second constituent material section 142 holding a second constituent material 150b and, optionally, one or more mixing masses 130. At step 608, the method 600 further includes, shaking (e.g., along an axial and/or transverse direction) the mixing syringe assembly 100 to combine the first constituent material with the second constituent material. After mixing and as described herein, the combined first and second constituent materials may be dispensed from the mixing syringe assembly such as by further advancing a plunger 102 of the embodiments described herein.

In embodiments including burstable films, piercing or rupturing the films may be accomplished via manual actuation or may be assisted with a jig or cradle and pressing actuator, (e.g. a linear actuator mechanically engaged with the plunger and/or the first barrel body portion).

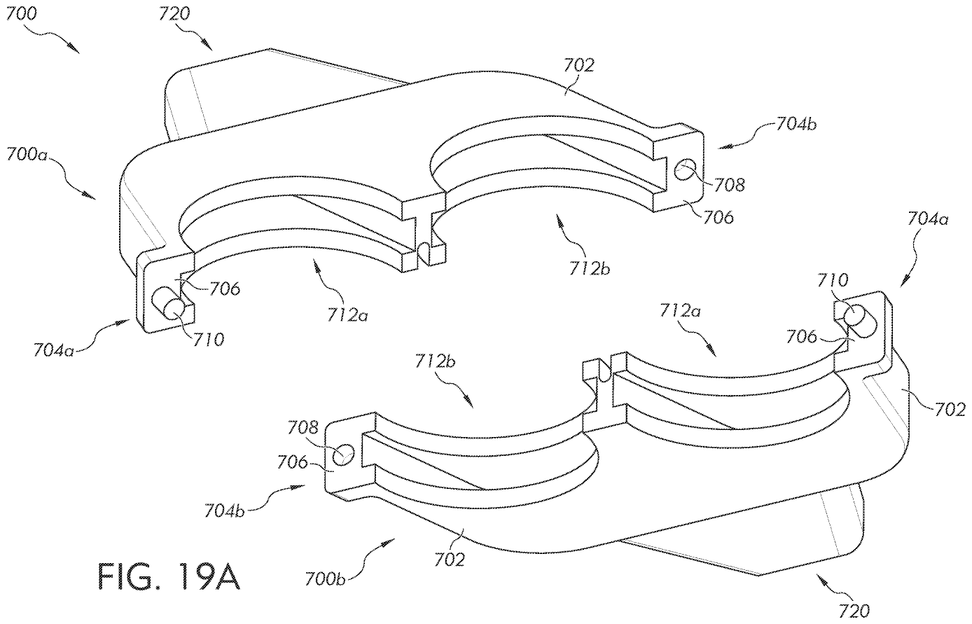

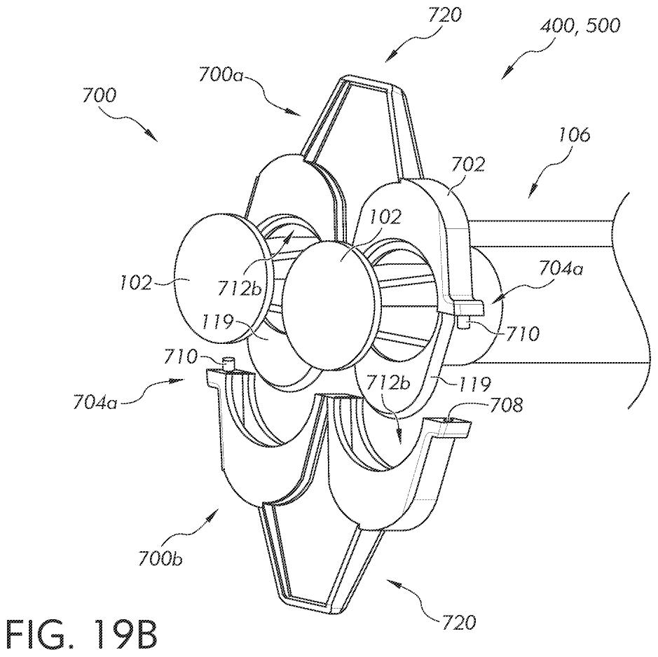

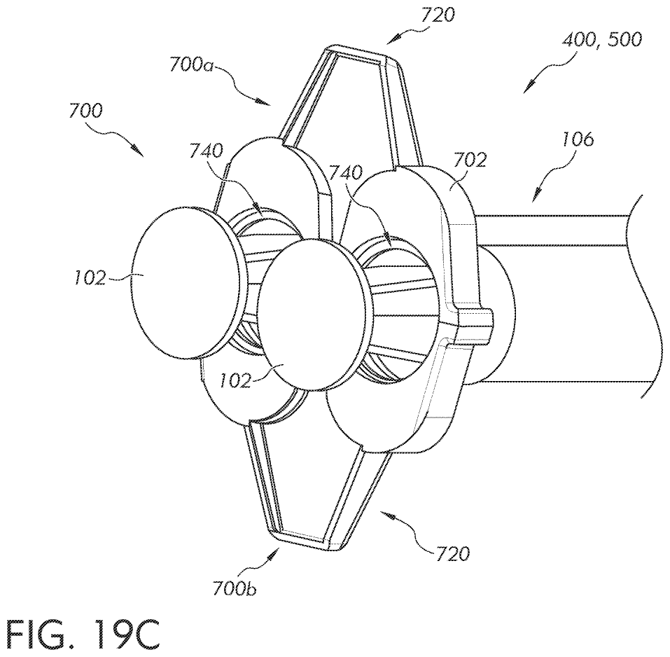

Referring to FIGS. 19A-19C, an illustrative two-piece alignment fixture 700 is depicted, which may be particularly useful in nested barrel embodiments described above, such as with respect to FIGS. 13A-17C, though it is contemplated that the two-piece alignment fixture 700 may be applicable to any nested barrel design and need not necessarily include all of the features described above. In particular, the two-piece alignment fixture 700 may be arranged around proximal ends of the smaller barrels, which allows for the smaller barrels to be bound together and enables a homogeneous distribution of pressure when hydrating the raw chemistry (e.g., powder/particulate portion) in the larger barrels. Accordingly, movement of the smaller barrel portions within the larger barrel portions are tied together via the fixture, making movement simultaneous. Additionally, the fixture may also provide a more comfortable and intuitive grip for a user during the hydration step.

In general, the two-piece alignment fixture 700 has a body 702 comprised of two parts: a first section 700a and a second section 700b. The two parts of the two-piece alignment fixture 700 may be substantially identical and/or mirror images of one another; as such, the various components for each will be numbered the same and described collectively for purposes of brevity.

The body 702 extends between a first end 704a and a second end 704b, and has a face 706. The face 706 includes various components that allow the two-piece alignment fixture 700 to be coupled to the applicator, such as a dual mixing syringe assembly described above. Particularly, the barrel body 106, such as first barrel body portions 406a, 506a, may each include a flange 119, see FIGS. 13A-13D and FIG. 16. The flanges 119 may be separate from one another. The two-piece alignment fixtures may extend around the flanges 119 such that the respective flanges 119 of each first barrel body portion 406a, 506a are coupled to one another. In some embodiments, the flanges 119 may already be coupled to one another and the fixture may provide additional gripping surface, for improved control/use.

To assist in coupling the two sections 700a, 700b together, the face 706 of the first section 700a and the second section 700b, such as at the first end 704a of the body 702 includes a protrusion 710, such as a detent, a knob, a post, or the like. The face 706 of the first section 700a and the second section 700b, such as at the second end 704b of the body 702 may include a mating recess 708. The protrusion 710 and the recess 708 may be complementary with respect to each other so that the protrusion 710 of the first section 700a fits within the recess 708 of the second section 700b, and the protrusion 710 of the second section 700b fits within the recess 708 of the first section 700a. In addition, the protrusion 710 and the recess 708 may be shaped and sized so that the first and second sections 700a, 700b are held together when joined. For example, the protrusion 710 may be flared, have retention pieces, or the like that provides a tight fit within the recess 708 when the first and second sections 700a, 700b are joined together.

To assist in in mounting the fixture 700 to the flanges 119 noted above, the face 706 may include a recess and/or a channel 712a, 712b that is shaped and sized to correspond to the flanges 119 of the first barrel body portions so that the flanges 119 are received within the recess and/or channel 712a, 712b when the first section 700a and the second section 700b are joined together around the flanges 119, as particularly depicted in FIG. 19C. As shown in FIG. 19C, tabs 720 extending from the body 702 of the two-piece alignment fixture 700 provide a location for a user to hold the assembly while depressing or retracting the plungers 102. When assembled, the fixture 700 provides an opening 740 therethrough for advancement/retraction of the plungers 102.

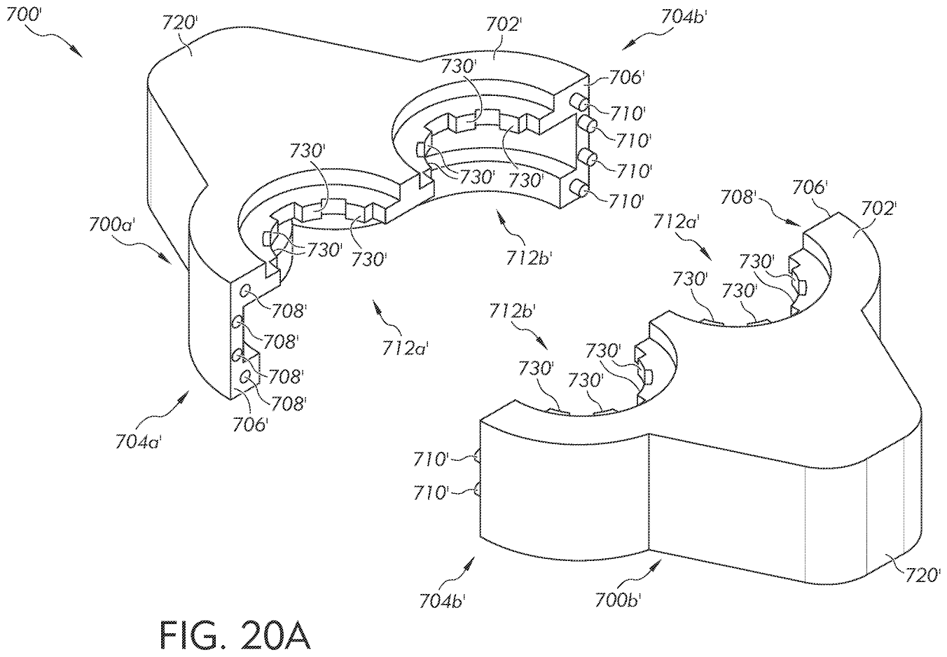

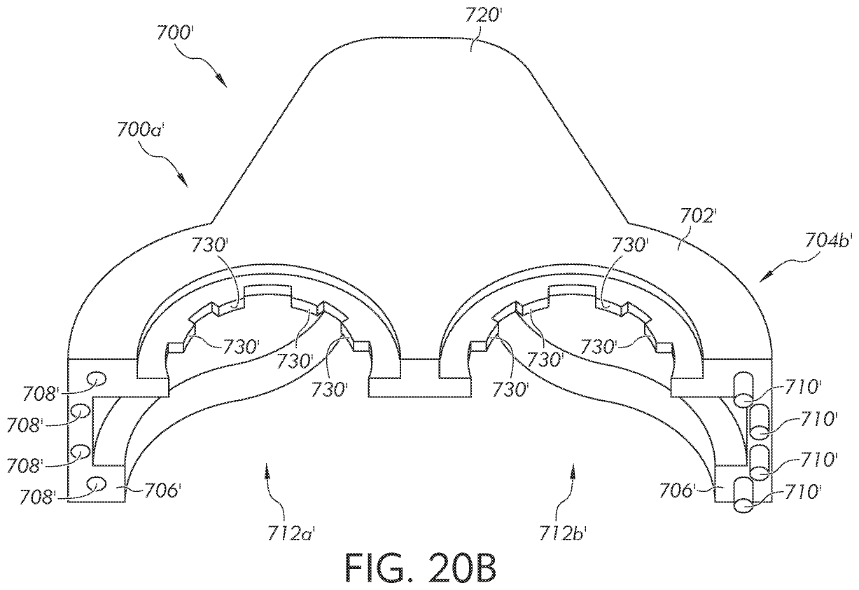

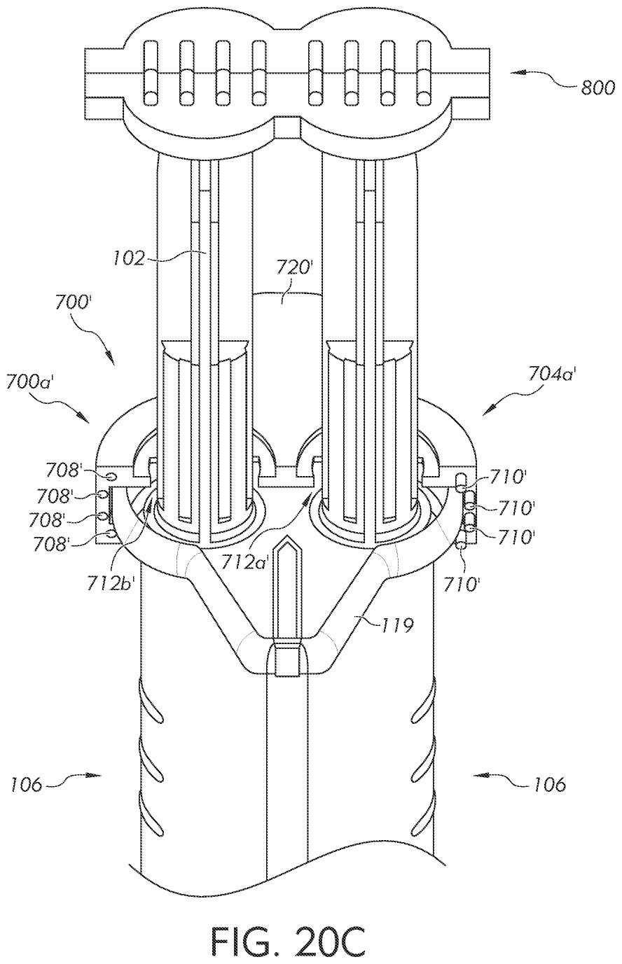

Referring to FIGS. 20A-20C, another two-piece alignment fixture 700′ is depicted, which may be used in place of alignment fixture 700 described above or as otherwise described. The two-piece alignment fixture 700′ has a body 702′ comprised of two parts: a first section 700a′ and a second section 700b′. The two parts of the two-piece alignment fixture 700′ may be substantially identical and/or mirror images of one another; as such, the various components for each will be numbered the same for purposes of brevity.

The body 702′ extends between a first end 704a′ and a second end 704b′, and has a face 706′. The face 706′ includes various components that allow the two-piece alignment fixture 700′ to be coupled to any of the mixing syringe assemblies as described herein, particularly the flanges thereof, such as a proximal flange of the barrel body 106 of the first barrel body portion 406, 506, as described above. For example, the face 706 at the first end 704a′ of the body 702′ includes a protrusion 710′, such as a detent, a knob, a post, or the like. In another example, the face 706′ at the second end 704b′ of the body 702′ includes a recess 708′. The protrusion 710′ and the recess 708′ may be complementary with respect to each other so that the protrusion 710′ of the first section 700a′ fits within the recess 708′ of the second section 700b′, and the protrusion 710′ of the second section 700b′ fits within the recess 708′ of the first section 700a′. In addition, the protrusion 710′ and the recess 708′ may be shaped and sized so that the first and second sections 700a′, 700b′ are held together when joined. For example, the protrusion 710′ may be flared, have retention pieces, or the like that provides a tight fit within the recess 708′ when the first and second sections 700a′, 700b′ are joined together. There may be any number of recesses/protrusions incorporated into the fixture 700′ to coupled the first and second sections 700a′, 700b′ to one another, such as a plurality thereof.

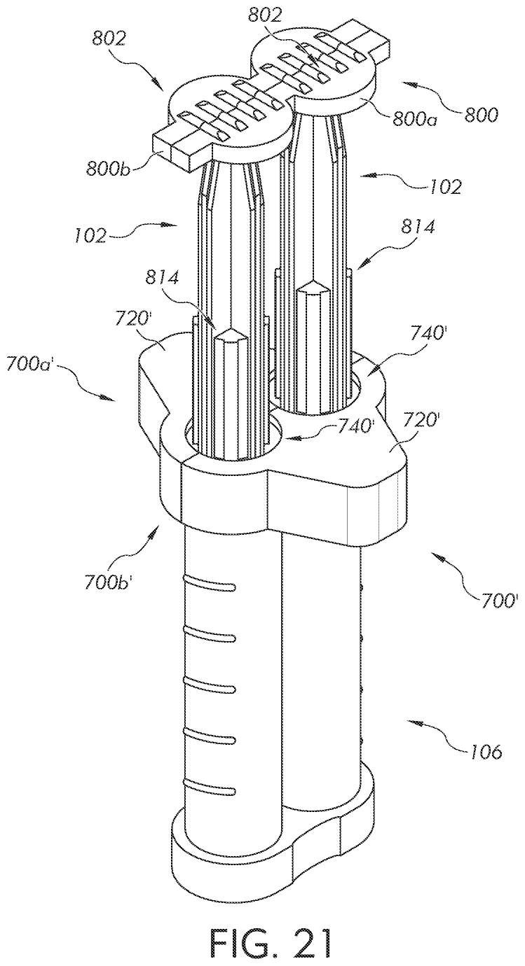

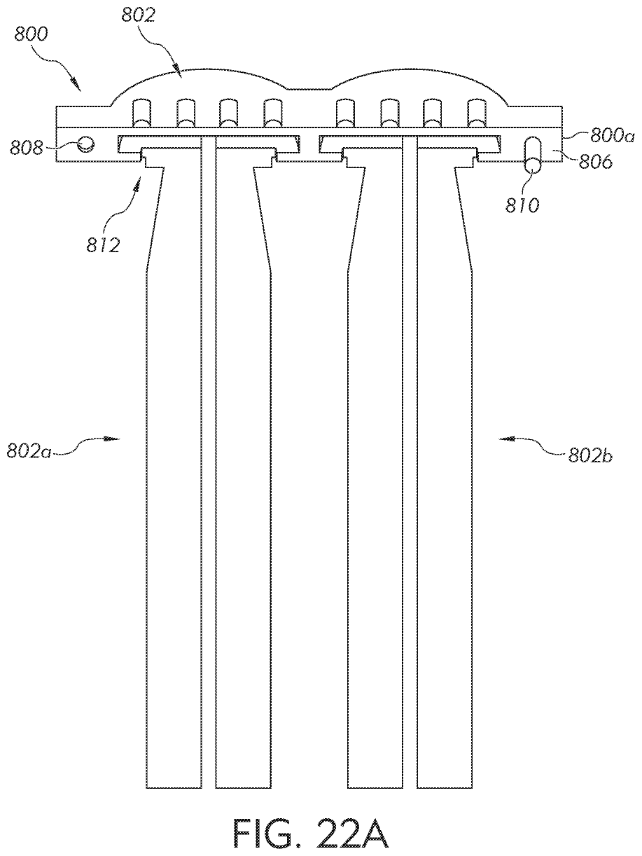

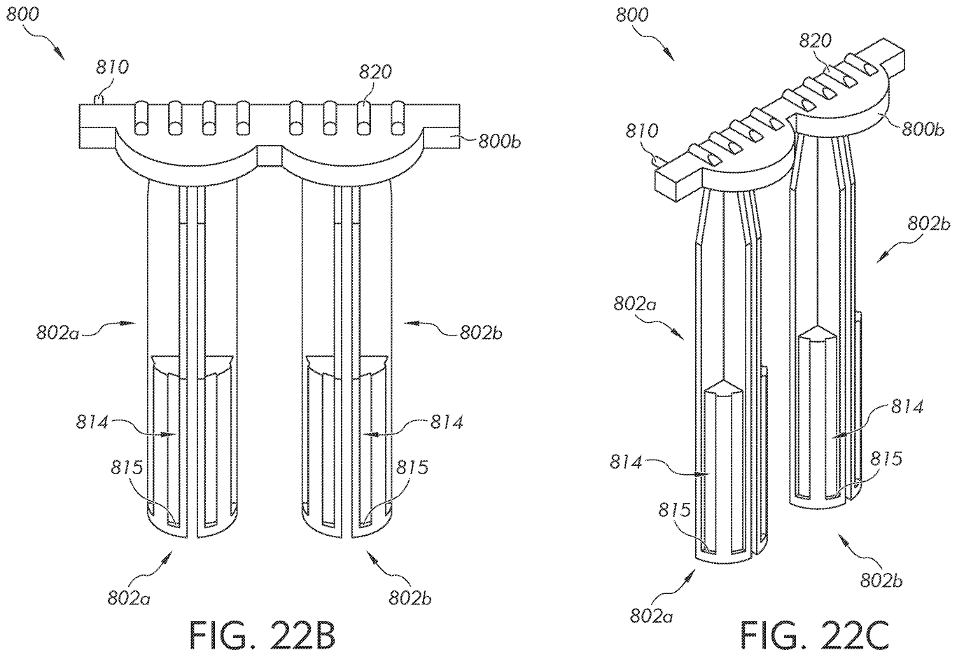

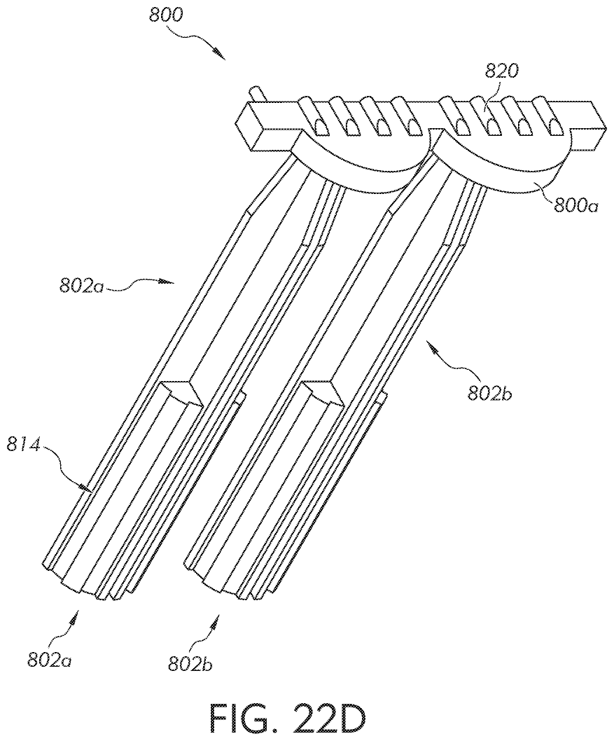

The face 706′ further includes a recess and/or a channel 712a′, 712b′ that is shaped and sized to correspond to the flange(s) 119 of the any of the mixing syringe assemblies (e.g., 100, 200, 300, 400, or 500) described herein, so that the flange(s) 119 are received within the recess and/or channel 712a′, 712b′ when the first section 700a′ and the second section 700b′ are joined together around the flange 119, as particularly depicted in FIG. 21C,-22. It is noted that FIG. 21C illustrates an embodiment where there is a common flange 119 connecting barrel bodies 106, though in embodiments, the flange s119 may be separate. As shown in FIG. 22, tabs 720′ extending from the body 702′ of the two-piece alignment fixture 700′ provide a location for a user to hold the mixing syringe assembly while depressing or retracting the plungers 102 of the respective first and second mixing syringe subassemblies.

As in the embodiment above, when assembled, the fixture 700′ provides an opening 740′ therethrough for advancement/retraction of the plungers 102. In the depicted embodiment the fixture 700′ may include protrusions 730′ or the like that extend radially into the opening 740′. Such protrusions 730′ are shaped and sized to fit within a corresponding channel of a two-piece piston insert 800, as described in greater detail herein.

Referring now to FIGS. 21-22D and 24, an illustrative two-piece piston insert 800 is depicted. The two-piece piston insert 800 has a body 802 comprised of two parts: a first section 800a and a second section 800b. The two parts of the two-piece piston insert 800 may be substantially identical and/or mirror images of one another; as such, the various components for each will be numbered the same for purposes of brevity. The two-piece piston insert 800 may act as a plunger connector connecting the plunger 102 of the first mixing syringe subassembly to the plunger 102 of the second mixing syringe subassembly, such that movement of one of the plungers 102 moves the other of the plungers 102.

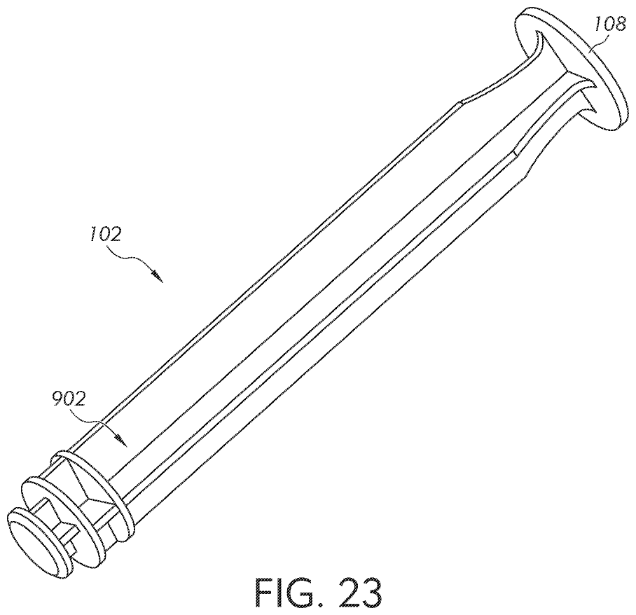

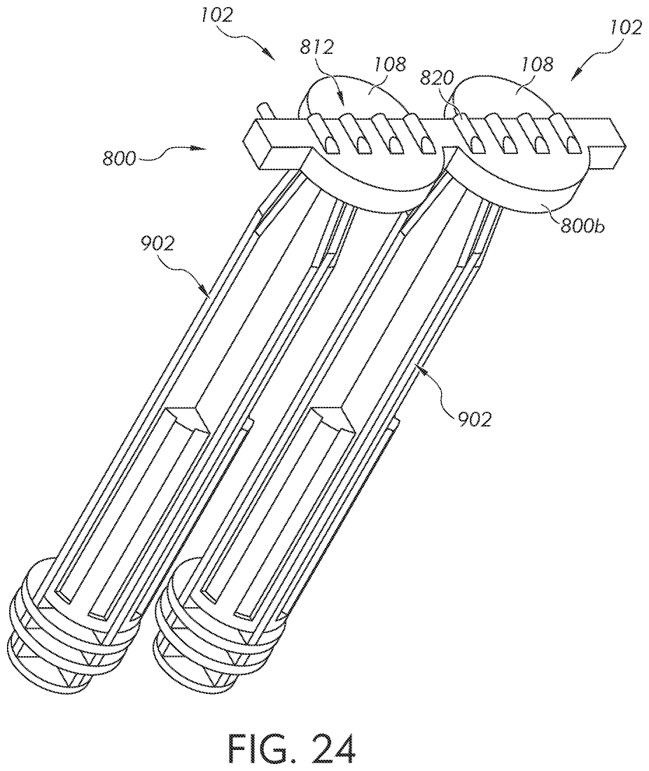

The body 802 extends between a first end and a second end, and has a face 806. A pair of extension pieces 802a, 802b extend distally from the body 802 and are molded to fit within grooves 902 of a corresponding plunger 102, as particularly shown in FIGS. 23 and 24. Accordingly, the pair of extension pieces 802a, 802b may be separated from one another via a channel. The face 806 includes various components that allow the two-piece piston insert 800 to be coupled to the plungers 102. For example, the face 806 at the first end of the body 802 includes a protrusion 810, such as a detent, a knob, a post, or the like. In another example, the face 806 at the second end of the body 802 includes a recess 808. The protrusion 810 and the recess 808 may be complementary with respect to each other so that the protrusion 810 of the first section 800a fits within the recess 808 of the second section 800b, and the protrusion 810 of the second section 800b fits within the recess 808 of the first section 800a. In addition, the protrusion 810 and the recess 808 may be shaped and sized so that the first and second sections 800a, 800b are held together when joined. For example, the protrusion 810 may be flared, have retention pieces, or the like that provides a tight fit within the recess 808 when the first and second sections 800a, 800b are joined together.

Other components of the face 806 include a recess and/or a channel 812 that is shaped and sized to correspond to the flanges 108 of the plunger 102 so that the flanges 108 are received within the recess and/or channel 812 when the first section 800a and the second section 800b are joined together around the plunger 102, as particularly depicted in FIG. 21. The resulting combination of the two-piece piston insert 800 and the plungers 102 is depicted in FIG. 21. In addition, as shown in FIGS. 21 and 24, surface features 820 extending from the body 802 of the two-piece piston insert 800 provide a location for a user to manipulate the plungers 102. The surface features 820 may include an array of raised bumps or ridges, though other surface features are contemplated and possible.

It should be understood that the two-piece piston insert 800 is an attachment device for a plunger 102 that provides increased surface area of the combined insert/plunger to be in contact with the inner wall of the barrel body 106, thereby providing increased stability. For example and as depicted, the two-piece piston insert 800 traces down the stem of the plunger 102 so as to be inserted in to the grooves 902 of the plunger 102. In embodiments, about half way down the combined insert/plunger 800/102 a radius is provided via the combination of the insert/plunger 102 matching that of the syringe barrel inner diameter (however this radius could be produced at any distance). The increased radius may assist in providing additional stability to the plungers 102. For example, as the plungers 102 are most distal from an end face of the barrel body 106, the two-piece piston insert 800 provides a stabilizer attachment that reduces perpendicular motion (from respect to the line of plunger 102 travel). This allows for a smoother deployment of the contents within the barrel body 106.

It is noted that in some embodiments, the two-piece piston insert 800 may not be included, instead features may be directly molded into the plunger 102, to minimize plunger wobbling during use.

In embodiments, the protrusions 730′ of the two-piece alignment fixture 700′ may engage with the two-piece piston insert 800. For example, the pair of extension pieces 802a, 802b may include longitudinal grooves 814 corresponding to the protrusions 730′ of the two-piece alignment fixture 700′. The longitudinal grooves 814 may end in a stop wall 815. When the plungers 102 and two-piece piston insert 800 are pulled proximally, the stop wall 815 may engage with the protrusions 730′ and prevent complete withdrawal of the plunger 102 and the two-piece piston insert 800 from the respective barrel bodies 106 and two-piece alignment fixture 700′. Accordingly, the plunger 102 and two-piece piston insert 800 may be locked into the assembly and the prevent accidental disassembly of the plunger 102 from the barrel body 106. In some embodiments, such as illustrated in FIG. 22D, there may be no stop wall.

It should now be understood that the present disclosure relates to various mixing syringe assemblies and methods of mixing constituent materials with mixing syringe assemblies. The various embodiments provided herein may provide ready to use or easily assembled syringe assemblies for easily mixing components with undue air bubble introduction. Moreover, embodiments as provided herein may assist in maintaining sterility and/or constituent material integrity, while improving ease of mixing and delivery.

Embodiments of the present disclosure may also be described with respect to the following numbered clauses:
1. A mixing syringe assembly for mixing two constituent materials, comprising: a barrel body; a plunger slidably positioned within the barrel body; a sealing device dividing the barrel body into a first constituent material section and a second constituent material section; and one or more mixing masses positioned within one of the first constituent material section and the second constituent material section, wherein the sealing device is operable to adjust from a sealing configuration to a non-sealing configuration to allow for a first constituent material from the first constituent material section to move into the second constituent material section upon movement of the plunger along the barrel body and be mixed therein with the one or more mixing masses.
2. The mixing syringe assembly of clause 1, wherein the sealing device comprises a one-way valve, wherein the one-way valve is configured to move from a closed configuration to an open configuration in response to the plunger moving closer to the sealing device.
3. The mixing syringe assembly of any preceding clause, wherein the sealing device comprises a gate valve configured to slide open in a radial direction relative to a centerline of the barrel body.
4. The mixing syringe assembly of any preceding clause, further comprising a protrusion extending inwardly from the barrel body toward a centerline of the barrel body, wherein: the sealing device comprises a longitudinally moveable seal; and movement of the plunger along the barrel body moves the longitudinally moveable seal over the protrusion, thereby releasing a seal of the longitudinally moveable seal with the barrel body.
5. The mixing syringe assembly of any preceding clause, wherein: the sealing device comprises one or more burstable films, and movement of the plunger along the barrel body causes the one or more burstable films to burst, thereby allowing the first constituent material to move from the first constituent material section to the second constituent material section.
6. The mixing syringe assembly of any preceding clause, wherein the sealing device comprises a capsule positioned at least partially within the second constituent material section, and comprising a first film and a second film the capsule configured to hold a second constituent material between the first film and the second film.
7. The mixing syringe assembly of any preceding clause, wherein the barrel body comprises a first barrel body portion and a second barrel body portion, wherein the first barrel body portion is slidably positioned within the second barrel body portion, and the first barrel body portion comprises a piercing nose coupled to the first barrel body portion, the piercing nose configured to pierce the first film and the second film upon movement of the first barrel body portion further within the second barrel body portion.
8. The mixing syringe assembly of any preceding clause, wherein: the barrel body comprises a first barrel body portion and a second barrel body portion, wherein the first barrel body portion is slidably positioned within the second barrel body portion, and the first barrel body portion comprises a piercing nose coupled to the first barrel body portion; the plunger is positioned within the first barrel body portion of the barrel body; and the sealing device is coupled to a distal end of the first barrel body portion of the barrel body within the second barrel body portion of the barrel body.
9. A mixing syringe assembly for mixing multiple constituent components, the mixing syringe assembly comprising: a first mixing syringe subassembly and a second mixing syringe subassembly coupled to the first mixing syringe subassembly, each mixing syringe subassembly comprising: a barrel body; a plunger slidably positioned within the barrel body; a sealing device dividing the barrel body into a first constituent material section and a second constituent material section; and one or more mixing masses positioned within one of the first constituent material section and the second constituent material section, wherein the sealing device is operable to adjust from a sealing configuration to a non-sealing configuration to allow for a first constituent material from the first constituent material section to move into the second constituent material section holding a second constituent material upon movement of the plunger along the barrel body and be mixed therein with the one or more mixing masses.
10. The mixing syringe assembly of clause 9, further comprising a fixture coupling the barrel body of the first mixing syringe subassembly and the barrel body of the second mixing syringe subassembly.
11. The mixing syringe of any preceding clause, further comprising a plunger connector connecting the plunger of the first mixing syringe subassembly to the plunger of the second mixing syringe subassembly, such that movement of one of the plungers moves the other of the plungers.
12. The mixing syringe assembly of any preceding clause, wherein the sealing device of at least one of the first mixing syringe subassembly and the second mixing syringe subassembly comprises a one-way valve, wherein the one-way valve is configured to move from a closed configuration to an open configuration in response to the plunger moving closer to the sealing device.
13. The mixing syringe assembly of any preceding clause, wherein the sealing device of at least one of the first mixing syringe subassembly and the second mixing syringe subassembly comprises a gate valve configured to slide open in a radial direction relative to a centerline of the barrel body.
14. The mixing syringe assembly of any preceding clause, wherein at least one of the first mixing syringe subassembly and the second mixing syringe subassembly further comprises a protrusion extending inwardly from the barrel body toward a centerline of the barrel body, wherein: the sealing device of the at least one of the first mixing syringe subassembly and the second mixing syringe subassembly comprises a longitudinally moveable seal; and movement of the plunger of the at least one of the first mixing syringe subassembly and the second mixing syringe subassembly along the barrel body moves the longitudinally moveable seal over the protrusion, thereby releasing a seal of the longitudinally moveable seal with the barrel body.
15. The mixing syringe assembly of any preceding clause, wherein: the sealing device of at least one of the first mixing syringe subassembly and the second mixing syringe subassembly comprises one or more burstable films, and movement of the plunger along the barrel body causes the one or more burstable films to burst, thereby allowing the first constituent material to move from the first constituent material section to the second constituent material section.
16. The mixing syringe assembly of any preceding clause, wherein the sealing device of at least one of the first mixing syringe subassembly and the second mixing syringe subassembly comprises one or more burstable films comprises a capsule positioned at least partially within the second constituent material section, and comprising a first film and a second film the capsule configured to hold the second constituent material between the first film and the second film.
17. The mixing syringe assembly of clause 16, wherein the barrel body of the at least one of the first mixing syringe subassembly and the second mixing syringe subassembly comprises a first barrel body portion and a second barrel body portion, wherein the first barrel body portion is slidably positioned within the second barrel body portion, and the first barrel body portion comprises a piercing nose coupled to the first barrel body portion, the piercing nose configured to pierce the first film and the second film upon movement of the first barrel body portion further within the second barrel body portion.
18. The mixing syringe assembly of any preceding clause, wherein: the barrel body of the first mixing syringe subassembly and the second mixing syringe subassembly comprises comprises a first barrel body portion and a second barrel body portion, wherein the first barrel body portion is slidably positioned within the second barrel body portion, and the first barrel body portion comprises a piercing nose coupled to the first barrel body portion; the plunger is positioned within the first barrel body portion of the barrel body; and the sealing device is coupled to a distal end of the first barrel body portion of the barrel body within the second barrel body portion of the barrel body.
19. A method of mixing constituent materials positioned within a mixing syringe assembly, the method comprising: advancing a plunger within a barrel body toward a sealing device separating the barrel body into a first constituent material section and a second constituent material section; adjusting the sealing device from a sealing configuration to a non-sealing configuration thereby providing a flow path between the first constituent material section and the second constituent material section; pushing with the plunger a first constituent material from the first constituent material section into the second constituent material section, the second constituent material section holding a second constituent material and one or more mixing masses; and shaking the mixing syringe assembly to combine the first constituent material with the second constituent material.
20. The method of any preceding clause, wherein the first constituent material is a fluid and the second constituent material is a powder.
21. A mixing syringe assembly for mixing two constituent materials, comprising: a barrel body comprising a first barrel body portion and a second barrel body portion, wherein the first barrel body portion is slidably disposed in the second barrel body portion, the first barrel body portion defining a first constituent material section and the second barrel body portion defining a second constituent material section; a plunger slidably positioned within the first barrel body portion; a sealing device comprising a piercing member coupled to a distal end of the first barrel body portion and a canister positioned within the second barrel body portion, wherein: the canister comprises a first pierceable film and a second pierceable film and a second constituent material is position within the canister between the first pierceable film and the second pierceable film; the piercing member is configured to pierce the first pierceable film and the second pierceable film to release the second constituent material; and a first constituent material is positioned within the first constituent material section and advancing the plunger releases the first constituent material in to the second constituent material section.
22. The mixing syringe assembly of any preceding clause, wherein the sealing device includes a hook device configured to engage with and couple to the canister.
23. The mixing syringe assembly of clause 22, wherein the hook device couples to the canister as the piercing member pierces the second pierceable film.
24. The mixing syringe assembly of clause 22 or 23, wherein the hook device comprises a plurality of radially outward facing hooks.
25. A mixing syringe assembly, comprising: a first mixing syringe subassembly and a second mixing syringe subassembly coupled to the first mixing syringe subassembly, each mixing syringe subassembly comprising: a first barrel body portion comprising a flange; a second barrel body portion, wherein the first barrel body portion is slidably disposed within the second barrel body portion; and a fixture coupled to the flange of the first mixing syringe subassembly and the second mixing syringe subassembly such that movement of the first barrel body portion of the first mixing syringe subassembly and the first barrel body portion of the second mixing syringe subassembly are tied to one another via the fixture.
26. The mixing syringe assembly of any preceding clause, wherein the fixture comprises a first section and a second section couplable to one another about the flange of the first barrel body portion of the first mixing syringe subassembly and the second barrel body portion of the second mixing syringe subassembly.
27. The mixing syringe assembly of any preceding clause, further comprising: a plunger slidably positioned within each of the first barrel body portions; and a two-piece piston insert coupled to each of the plungers, wherein the two-piece piston insert engages with the fixture to prevent withdrawal of the plungers from the first barrel body portions.
28. A mixing syringe assembly, comprising: a first mixing syringe subassembly and a second mixing syringe subassembly coupled to the first mixing syringe subassembly, each mixing syringe subassembly comprising: a barrel body comprising a flange; and a plunger slidably positioned within each of the first barrel body portions; a fixture coupled to the flange of the first mixing syringe subassembly and the second mixing syringe subassembly; and a two-piece piston insert coupled to each of the plungers, wherein the two-piece piston insert engages with the fixture to prevent withdrawal of the plungers from the first barrel body portions.
29. The mixing syringe assembly of clause 28, wherein the fixture comprises a first section and a second section couplable to one another about the flange of the first mixing syringe subassembly and the flange of the second mixing syringe subassembly.

While particular embodiments have been illustrated and described herein, it should be understood that various other changes and modifications may be made without departing from the spirit and scope of the claimed subject matter. Moreover, although various aspects of the claimed subject matter have been described herein, such aspects need not be utilized in combination. It is therefore intended that the appended claims cover all such changes and modifications that are within the scope of the claimed subject matter.

1. A mixing syringe assembly for mixing two constituent materials, comprising:
a barrel body;
a plunger slidably positioned within the barrel body;
a sealing device dividing the barrel body into a first constituent material section and a second constituent material section; and
one or more mixing masses positioned within one of the first constituent material section and the second constituent material section,
wherein the sealing device is operable to adjust from a sealing configuration to a non-sealing configuration to allow for a first constituent material from the first constituent material section to move into the second constituent material section upon movement of the plunger along the barrel body and be mixed therein with the one or more mixing masses.

2. The mixing syringe assembly of claim 1, wherein the sealing device comprises a one-way valve, wherein the one-way valve is configured to move from a closed configuration to an open configuration in response to the plunger moving closer to the sealing device.

3. The mixing syringe assembly of claim 1, wherein the sealing device comprises a gate valve configured to slide open in a radial direction relative to a centerline of the barrel body.

4. The mixing syringe assembly of claim 1, further comprising a protrusion extending inwardly from the barrel body toward a centerline of the barrel body, wherein:
the sealing device comprises a longitudinally moveable seal; and
movement of the plunger along the barrel body moves the longitudinally moveable seal over the protrusion, thereby releasing a seal of the longitudinally moveable seal with the barrel body.

5. The mixing syringe assembly of claim 1, wherein:
the sealing device comprises one or more burstable films, and
movement of the plunger along the barrel body causes the one or more burstable films to burst, thereby allowing the first constituent material to move from the first constituent material section to the second constituent material section.

6. The mixing syringe assembly of claim 1, wherein the sealing device comprises a capsule positioned at least partially within the second constituent material section, and comprising a first film and a second film the capsule configured to hold a second constituent material between the first film and the second film.

7. The mixing syringe assembly of claim 6, wherein the barrel body comprises a first barrel body portion and a second barrel body portion, wherein the first barrel body portion is slidably positioned within the second barrel body portion, and the first barrel body portion comprises a piercing nose coupled to the first barrel body portion, the piercing nose configured to pierce the first film and the second film upon movement of the first barrel body portion further within the second barrel body portion.

8. The mixing syringe assembly of claim 1, wherein:
the barrel body comprises a first barrel body portion and a second barrel body portion, wherein the first barrel body portion is slidably positioned within the second barrel body portion, and the first barrel body portion comprises a piercing nose coupled to the first barrel body portion,
the plunger is positioned within the first barrel body portion of the barrel body; and
the sealing device is coupled to a distal end of the first barrel body portion of the barrel body within the second barrel body portion of the barrel body.

9. A mixing syringe assembly for mixing multiple constituent components, the mixing syringe assembly comprising:
a first mixing syringe subassembly and a second mixing syringe subassembly coupled to the first mixing syringe subassembly, each mixing syringe subassembly comprising:
a barrel body;
a plunger slidably positioned within the barrel body;
a sealing device dividing the barrel body into a first constituent material section and a second constituent material section; and
one or more mixing masses positioned within one of the first constituent material section and the second constituent material section,
wherein the sealing device is operable to adjust from a sealing configuration to a non-sealing configuration to allow for a first constituent material from the first constituent material section to move into the second constituent material section holding a second constituent material upon movement of the plunger along the barrel body and be mixed therein with the one or more mixing masses.

10. The mixing syringe assembly of claim 9, further comprising a fixture coupling the barrel body of the first mixing syringe subassembly and the barrel body of the second mixing syringe subassembly.

11. The mixing syringe of claim 9, further comprising a plunger connector connecting the plunger of the first mixing syringe subassembly to the plunger of the second mixing syringe subassembly, such that movement of one of the plungers moves the other of the plungers.

12. The mixing syringe assembly of claim 9, wherein the sealing device of at least one of the first mixing syringe subassembly and the second mixing syringe subassembly comprises a one-way valve, wherein the one-way valve is configured to move from a closed configuration to an open configuration in response to the plunger moving closer to the sealing device.

13. The mixing syringe assembly of claim 9, wherein the sealing device of at least one of the first mixing syringe subassembly and the second mixing syringe subassembly comprises a gate valve configured to slide open in a radial direction relative to a centerline of the barrel body.

14. The mixing syringe assembly of claim 9, wherein at least one of the first mixing syringe subassembly and the second mixing syringe subassembly further comprises a protrusion extending inwardly from the barrel body toward a centerline of the barrel body, wherein:
the sealing device of the at least one of the first mixing syringe subassembly and the second mixing syringe subassembly comprises a longitudinally moveable seal; and
movement of the plunger of the at least one of the first mixing syringe subassembly and the second mixing syringe subassembly along the barrel body moves the longitudinally moveable seal over the protrusion, thereby releasing a seal of the longitudinally moveable seal with the barrel body.

15. The mixing syringe assembly of claim 9, wherein:
the sealing device of at least one of the first mixing syringe subassembly and the second mixing syringe subassembly comprises one or more burstable films, and
movement of the plunger along the barrel body causes the one or more burstable films to burst, thereby allowing the first constituent material to move from the first constituent material section to the second constituent material section.

16. The mixing syringe assembly of claim 9, wherein the sealing device of at least one of the first mixing syringe subassembly and the second mixing syringe subassembly comprises one or more burstable films comprises a capsule positioned at least partially within the second constituent material section, and comprising a first film and a second film the capsule configured to hold the second constituent material between the first film and the second film.

17. The mixing syringe assembly of claim 16, wherein the barrel body of the at least one of the first mixing syringe subassembly and the second mixing syringe subassembly comprises a first barrel body portion and a second barrel body portion, wherein the first barrel body portion is slidably positioned within the second barrel body portion, and the first barrel body portion comprises a piercing nose coupled to the first barrel body portion, the piercing nose configured to pierce the first film and the second film upon movement of the first barrel body portion further within the second barrel body portion.

18. The mixing syringe assembly of claim 9, wherein:
the barrel body of the first mixing syringe subassembly and the second mixing syringe subassembly comprises a first barrel body portion and a second barrel body portion, wherein the first barrel body portion is slidably positioned within the second barrel body portion, and the first barrel body portion comprises a piercing nose coupled to the first barrel body portion,
the plunger is positioned within the first barrel body portion of the barrel body; and
the sealing device is coupled to a distal end of the first barrel body portion of the barrel body within the second barrel body portion of the barrel body.

19. A method of mixing constituent materials positioned within a mixing syringe assembly, the method comprising:
advancing a plunger within a barrel body toward a sealing device separating the barrel body into a first constituent material section and a second constituent material section;
adjusting the sealing device from a sealing configuration to a non-sealing configuration thereby providing a flow path between the first constituent material section and the second constituent material section;
pushing with the plunger a first constituent material from the first constituent material section into the second constituent material section, the second constituent material section holding a second constituent material and one or more mixing masses; and
shaking the mixing syringe assembly to combine the first constituent material with the second constituent material.

20. The method of claim 19, wherein the first constituent material is a fluid and the second constituent material is a powder.

21-29. (canceled)
